(19) United States
(12) Patent Application Publication
(10) Pub. No.: US 2015/0157290 A1
(43) Pub. Date: Jun. 11, 2015

(54) INFORMATION PROCESSING DEVICE, INFORMATION PROCESSING METHOD, CONTROL PROGRAM AND RECORDING MEDIUM

(71) Applicant: Sharp Kabushiki Kaisha, Osaka-shi, Osaka (JP)

(72) Inventors: Tomohisa Kawata, Osaka-shi (JP); Yutaka Ikeda, Osaka-shi (JP)

(21) Appl. No.: 14/402,783

(22) PCT Filed: Apr. 23, 2013
(86) PCT No.: PCT/JP2013/061931

§ 371 Date: Nov. 21, 2014

(30) Foreign Application Priority Data
Jun. 21, 2012 (JP) 2012-140131
Aug. 3, 2012 (JP) 2012-173435

(51) Int. Cl. A61B 7/04; A61B 5/00

(52) U.S. Cl. CPC A61B 7/04; A61B 5/7221; A61B 5/742

(57) ABSTRACT

A terminal device (100) includes an abnormality determination unit (111), a recording process unit (112), and a display control unit (113). The display control unit (113) displays an indication screen for indicating observation positions on which a stethoscope (30) is to be placed to an operator (U) of the stethoscope (30) on a display unit (104). The recording process unit (112) performs a biological sound observation process for recording on a storage unit (103) by associating positional information that shows observation positions with biological sound information that is output from the stethoscope 30. The abnormality determination unit (111) determines whether or not the biological sound information is an abnormality candidate. The display control unit (113) displays the indication screen such that the biological sound observation process is executed at least twice for each of observation positions of biological sound information that are determined to be an abnormality candidate, and bilateral positions, which have a bilaterally symmetrical relationship with respect to the observation positions.

### TECHNICAL FIELD

The present invention relates to an information processing device that processes biological sound information that is acquired by an electronic stethoscope, an information processing method, a control program and recording medium.

### BACKGROUND ART

Electronic stethoscopes that acquire biological sounds (respiratory system sounds, cardiac sounds and the like) from biological objects (patients), and output the acquired biological sounds as digital signals (biological sound information), are in widespread use. In addition, information processing devices having a function that determines whether or not the biological sound information or the state of the patient is an abnormality candidate (has a possibility of abnormality) by analyzing the biological sound information that is output from the electronic stethoscope, are also in widespread use, and the inventions disclosed in PTL 1 to PTL 3 are known as examples of devices that have this kind of function.

In addition, since it is possible to record biological sound information that is obtained by electronic stethoscopes, it is possible to realize a diagnosis state in which the diagnosis of a health condition of a patient is performed by a user (an operator) other than a doctor recording biological sound information through auscultation of the patient using an electronic stethoscope, and a doctor subsequently reproducing and listening to the biological sound information. In this kind of diagnosis state, since a user other than a doctor performs observation, systems having a function that indicates an observation method to a user by displaying observation positions and a sequence of observation of each observation position, have been developed. It is possible to include the inventions that are disclosed in PTL 4 and PTL 5 as examples of such systems.

### CITATION LIST

### Patent Literature

PTL 1: Japanese Unexamined Patent Application Publication No. 2005-66044 (Mar. 17, 2005)

PTL 2: Japanese Unexamined Patent Application Publication No. 2007-190082 (Aug. 2, 2007)

PTL 3: Japanese Unexamined Patent Application Publication No. 2008-113936 (May 22, 2008)

PTL 4: Japanese Unexamined Patent Application Publication No. 2001-327488 (Nov. 27, 2001)

PTL 5: Japanese Unexamined Patent Application Publication (Translation of PCT application) No. 2010-525363 (Jul. 22, 2010)

### SUMMARY OF INVENTION

### Technical Problem

However, according to a diagnosis system that uses an electronic stethoscope, there is a possibility that it will not be possible to perform high-precision diagnosis as a result of either the first cause or the second cause below.

(First Cause)

Firstly, the first cause will be described. In a case where a doctor performs diagnosis at a given observation position, rather than diagnosis being performed from biological sounds of the corresponding observation position only, diagnosis is performed by taking a comparison result of biological sounds of the corresponding observation position and biological sounds of an observation position that has a bilaterally symmetrical positional relationship with respect to the corresponding observation position (hereinafter referred to as a “symmetrical position”) into consideration. This is because, in order to perform accurate diagnosis at a given observation position, it is not sufficient to use biological sounds of the corresponding observation position only as an absolute assessment, and therefore, it is also necessary to perform a relative assessment by comparing the biological sounds of the corresponding observation position and biological sounds of a symmetrical position.

Furthermore, in a case where a doctor listens to biological sounds that are detected from a given observation position, and thinks that there is a suspicion of an abnormality, rather than concluding diagnosis with a single auscultation of each of the corresponding observation position and the symmetrical position, there is a tendency to improve the precision of the diagnosis through repeated auscultation. This is because, in a single auscultation, there is also a chance that there are circumstances in which the biological sounds deviate from normal sounds or the like due to mere noise or the like.

However, in a case where a user of an electronic stethoscope is a person other than a doctor, since there are many circumstances in which the user cannot determine whether or not there is a suspicion of an abnormality in the first place, it is possible to think circumstances in which the user concludes diagnosis with a single auscultation even at observation positions that would normally be determined as abnormal in a case where a doctor was performing auscultation. For this reason, even if a doctor subsequently reproduces the biological sound information of the corresponding observation position and thinks that there is a suspicion of an abnormality, since only data for a single auscultation has been stored for each of the corresponding observation position and the symmetrical position, there is a possibility that it will not be possible for the doctor to perform high-precision diagnosis at a later time.

That is, in a case where a user (an auscultator) of an electronic stethoscope is a person other than a doctor, and a doctor performs diagnosis by subsequently reproducing recorded data, there is a possibility that the precision of the diagnosis will be reduced due to a first cause described above, since the auscultator is a person other than a doctor, diagnosis may be concluded with a single auscultation even at observation positions that would be determined as abnormal if a doctor was performing auscultation themselves.

(Second Cause)

Next, the second cause will be described. In a case where a doctor performs diagnosis at a given observation position themselves using an electronic stethoscope, rather than performing diagnosis from the biological sounds of the corresponding observation position only, diagnosis is performed by comparing biological sounds of the corresponding observation position and biological sounds of an observation position that has a bilaterally symmetrical positional relationship with respect to the corresponding observation position (hereinafter referred to as a “symmetrical position”). This is because, it is thought that there is a possibility of some kind of abnormality if the biological sounds are not similar.

In addition, in a case where a doctor listens to and compares biological sounds that are obtained by performing a single observation respectively at a given observation position and a symmetrical position and determined that the biological sounds are not similar, it is normal to improve the precision of the diagnosis by attempting to listen to biological sounds with a different timing to those of the first observation, and reexamining whether or not the biological sounds are not similar, thereby performing re-observation at each of the corresponding observation position and the symmetrical position. This is because, in a single auscultation, there is also a chance that there are circumstances in which the biological sounds are not similar due to mere noise or the like.

However, in a case where a user of an electronic stethoscope is a person other than a doctor, since there are many circumstances in which the user does not listen to and compare the biological sounds of the observation position and the biological sounds of the symmetrical position carefully, there are many cases in which re-observation is not performed even when the sets of biological sounds are not similar. For this reason, even if a doctor subsequently reproduces the biological sound information of the given observation position and the symmetrical position and thinks that the sets of biological sounds are not similar, it is not possible to listen to biological sound information of a re-observation since only data for a single auscultation has been stored for each of the corresponding observation position and the symmetrical position, and therefore, there is a possibility that it will not be possible for the doctor to perform high-precision diagnosis at a later time.

That is, in a case where a user (an auscultator) of an electronic stethoscope is a person other than a doctor, and a doctor performs diagnosis by subsequently reproducing recorded data, there is a possibility that the precision of the diagnosis will be reduced due to a second cause described above, since the auscultator is a person other than a doctor, diagnosis may be concluded with a single auscultation even at observation positions and a symmetrical position, the biological sounds of which would be determined as dissimilar if a doctor was performing auscultation themselves.

### OBJECT OF THE INVENTION

A first object of the present invention is to provide an information processing device that is capable of suppressing reductions in the precision of the diagnosis by suppressing the first cause described above.

In addition, a second object of the present invention is to provide an information processing device that is capable of suppressing reductions in the precision of the diagnosis by suppressing the second cause described above.

### Solution to Problem

In order to achieve the above-described first object, an information processing device according to a first embodiment of the present invention includes an indication unit that indicates observation positions on which a stethoscope is to be placed to a user of the stethoscope, a recording process unit that performs a biological sound observation process, which is a process for recording on a storage device by associating positional information that shows observation positions indicated by the indication unit with biological sound information that is received by the stethoscope, and an abnormality determination unit that determines whether or not the biological sound information is an abnormality candidate. When the indication unit sets an observation position that is shown by positional information which is associated with biological sound information that is determined to be an abnormality candidate as an abnormality observation position, and sets an observation position, which has a paired relationship with the abnormality observation position according to a predetermined rule, as a predetermined observation position in a case where it is determined in the abnormality determination unit that the biological sound information is an abnormality candidate, the indication unit performs the indication such that the biological sound observation process is executed at least twice for each of the abnormality observation position and the predetermined observation position.

In addition, in order to achieve the above-described second object, an information processing device according to a second embodiment of the present invention includes an indication unit that executes an indication process indicating observation positions on which a stethoscope is to be placed to a user of the stethoscope, a recording process unit that performs a biological sound observation process, which is a process for recording on a storage device by associating positional information that shows observation positions which are indicated by the indication unit with biological sound information that is received by the stethoscope, and a dissimilarity determination unit that performs dissimilarity determination of whether or not a waveform of first biological sound information that is stored in the storage device in association with positional information of a first observation position, and a waveform of second biological sound information that is stored in the storage device in association with a second observation position, which is an observation position that has a paired relationship with the first observation position according to a predetermined rule, are not similar. In a case where it is determined by the dissimilarity determination that the first biological sound information and the second biological sound information are not similar, the indication unit is configured to perform the indication process such that the biological sound observation process is performed again for each of the first observation position and the second observation position.

### Advantageous Effects of Invention

According to the above-described first embodiment of the present invention, in a case where biological sound information for which there is a suspicion of an abnormality, is acquired (a case in which the biological sound information is determined to be an abnormality candidate), the biological sound observation process is performed at least twice for each of the abnormality observation position, which is an acquisition source of the corresponding biological sound information, and the predetermined observation position (a symmetrical position), which has a paired relationship with the abnormality observation position according to a predetermined rule. Therefore, since a doctor, who subsequently performs diagnosis by reproducing the biological sound information, has the opportunity to listen to at least two items of biological sound information for each of the abnormality observation position for which there is a suspicion of an abnormality and the predetermined observation position, there is an effect of being capable of improving the precision of the diagnosis. In other words, by suppressing the above-described first cause, it is possible to suppress reductions in the precision of the diagnosis.

In addition, according to the above-described second embodiment of the present invention, in a case where there is a possibility that the biological sound information of the first observation position and the biological sound information of the second observation position (a position that is bilaterally symmetrical to the first observation position), which has a paired relationship with the first observation position, are not similar, it is possible to reproduce the biological sound information of at least two auscultations for each of the first observation position and the second observation position, and therefore, there is an effect of being capable of improving the precision of the diagnosis by a doctor or the like. In other words, by suppressing the above-described second cause, it is possible to suppress reductions in the precision of the diagnosis.

### BRIEF DESCRIPTION OF DRAWINGS

FIG. 1 is a block diagram that shows a configuration of a terminal device in Embodiment 1.

FIG. 2 is an explanatory diagram that describes an outline of an auscultation system that is provided with the terminal device in FIG. 1.

FIG. 3 illustrates diagrams that show observation numbers that specify each observation position and a sequence of observation of each observation position. Additionally, FIG. 3(a) is a diagram of a case of a dorsal region mode, and FIG. 3(b) is a diagram of a case of a precordial region mode.

FIG. 4 is an explanatory diagram that shows an example of an observation table that is stored in a storage unit that is shown in FIG. 1.

FIG. 5 illustrates diagrams that show examples of indication screens that are displayed on a display unit which the terminal device in FIG. 1 is provided with.

FIG. 6 illustrates diagrams that show examples of indication screens that are displayed on a display unit which the terminal device in FIG. 1 is provided with, and are diagrams that show indication screens that are displayed after the indication screens in FIG. 5 are displayed.

FIG. 7 is a flowchart that shows a process flow in the auscultation system in FIG. 2.

FIG. 8 illustrates diagrams that show indication screens in a first modification example.

FIG. 9 is a diagram that shows an indication screens in a second modification example.

FIG. 10 is a block diagram that shows a configuration of a terminal device in Embodiment 2.

FIG. 11 is an explanatory diagram that shows an example of an observation table that is stored in a storage unit that is shown in FIG. 10.

FIG. 12 illustrates diagrams that show examples of indication screens that are displayed on a display unit which the terminal device in FIG. 10 is provided with.

FIG. 13 illustrates diagrams that show examples of indication screens that are displayed on a display unit which the terminal device in FIG. 10 is provided with, and are diagrams of screens that are displayed after the indication screen of FIG. 12(f) is displayed.

FIG. 14 illustrates diagrams that show examples of indication screens that are displayed on a display unit which the terminal device in FIG. 10 is provided with, and are diagrams of screens that are displayed after the indication screen of FIG. 13(f) is displayed.

FIG. 15 is a flowchart that shows a process flow of Embodiment 2.

### DESCRIPTION OF EMBODIMENTS

### Embodiment 1

Firstly, an outline of an auscultation system (an information processing system), which is an embodiment of the present invention will be described with reference to the drawings.

(Outline of Auscultation System)

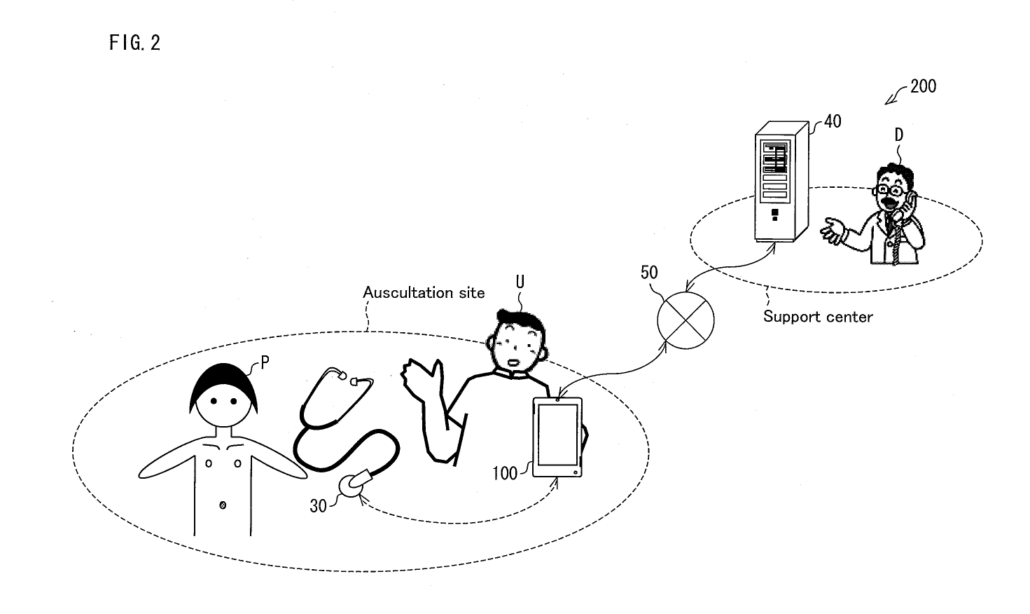

FIG. 2 is a diagram that shows an outline of auscultation system 200 of the present embodiment. As shown in FIG. 2, the auscultation system 200 includes a stethoscope (an electronic stethoscope) 30, a terminal device 100 and a server device 40. The stethoscope 30 and the terminal device 100 are used at a medical care site, and the server device 40 is a computer that is separated from the medical care site, and that is installed in a support center where doctors are stationed.

The stethoscope 30 and the terminal device 100 are communicably connected to one another in either a wired or a wireless manner, and the terminal device 100 and the server device 40 are communicably connected with one another through a network 50 such as the Internet.

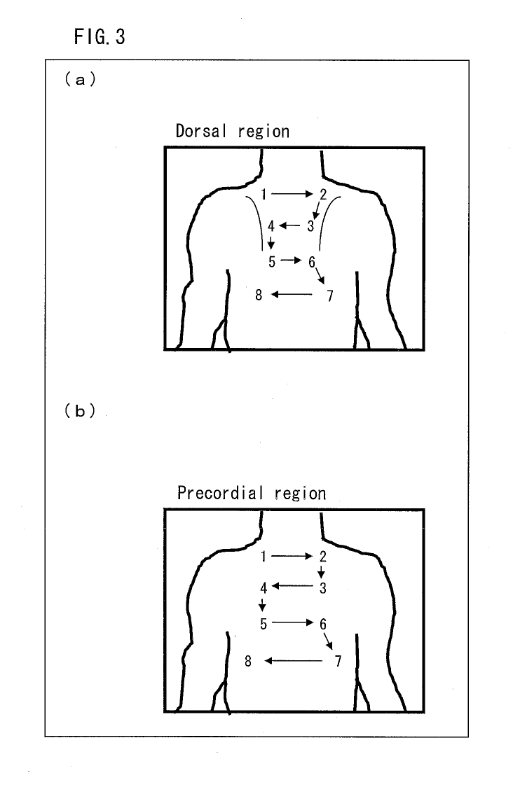

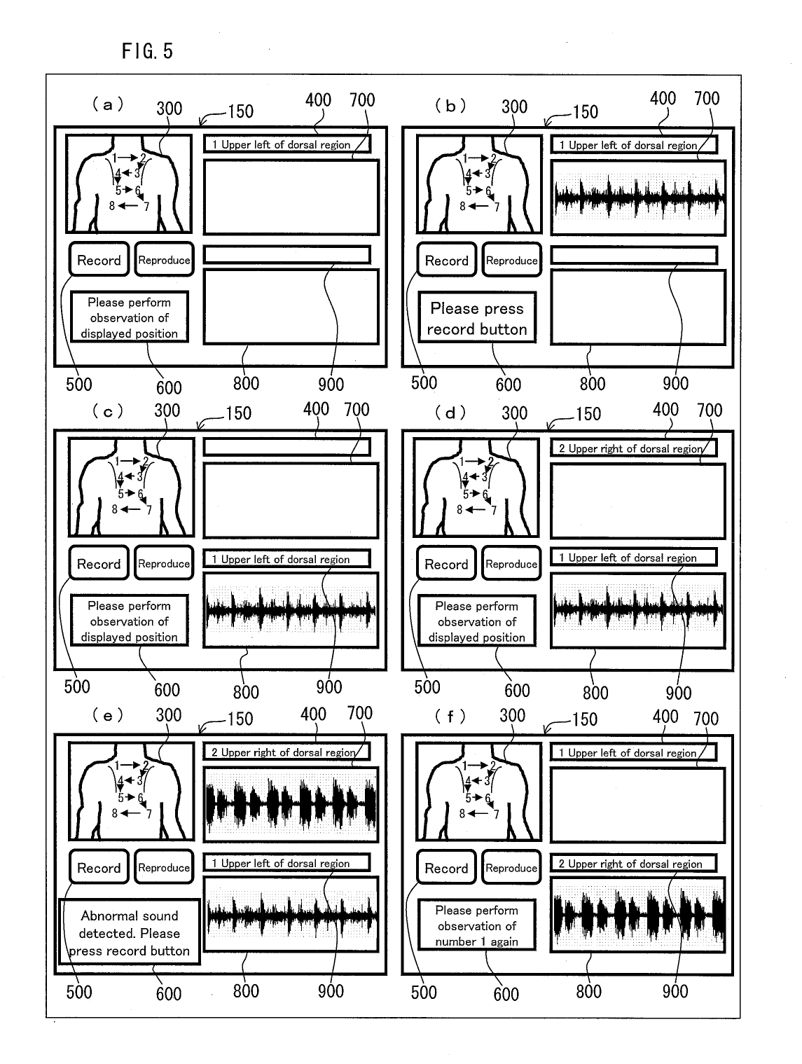

In the auscultation system 200 of the present embodiment, auscultation is performed in the following manner. At the medical care site that is shown in FIG. 2, an operator U displays an indication screen (refer to FIG. 5) that indicates a sequence of observation on a terminal device 100, and performs auscultation on a patient P using the stethoscope 30 according to the content of the indication screen. More specifically, the operator U auscultates observation positions (1 to 8 that are shown in FIG. 3(a)) that are designated on an indication screen. At this time, biological sound information that is acquired by the stethoscope 30 is transmitted from the stethoscope 30 to the terminal device 100, and the terminal device 100 determines whether or not the biological sound information during auscultation is an abnormality candidate (a sound that has a high probability of being an abnormal sound).

Further, when the operator U presses a record button on the indication screen while auscultating the observation position, the terminal device 100 is configured to save by linking abnormality information (abnormality sound information) that shows whether or not the biological sound information during auscultation is an abnormality candidate (a sound that has a high probability of being an abnormal sound), the corresponding biological sound information, and observation position information (an observation number that will be described later) that shows the observation position of the corresponding biological sound information. When recording of the biological sound information by the terminal device 100 is completed, and the operator U completes auscultation of the observation position that corresponds to the corresponding biological sound information, the terminal device 100 displays (designates) a subsequent observation position which should be observed on the indication screen. Auscultation of all of the observation position (1 to 8 in FIG. 3(a)) which are established in advance is performed by repeating a procedure such as that described above, and the biological sound information of each observation position is saved in the terminal device 100.

Subsequently, the biological sound information that is saved in the terminal device 100 and each kind of information that is linked to the corresponding biological sound information is sent to the server device 40 due to operation of the terminal device 100 by the operator U or operation of the server device 40 by a doctor D. While confirming the observation position information (the observation number that will be described later) which is linked to the biological sound information, the doctor D reproduces and listens to the biological sound information that is linked to the corresponding observation position information, and thereby it is possible to perform diagnosis of the patient P.

Additionally, the operator U may be a person skilled in the art other than a doctor. For example, it is possible to include a healthcare professional such as a registered nurse, or a professional caregiver such as a certified care worker as examples of the operator U.

(Configuration of Auscultator 30)

The stethoscope 30 is a so-called electronic stethoscope. That is, it is possible to acquire biological sounds from a patient P, who is an observation target, by applying a chestpiece of the stethoscope 30 to either a chest region mode or a dorsal region of the patient P. The kind of biological sound is not particularly limited, but for example, may be cardiac sounds, respiratory sounds or intestinal sounds. The acquired sounds are A/D converted, sent to the terminal device 100 as digital biological sound information, and managed by the terminal device 100.

(Configuration of Terminal Device 100)

The terminal device 100 is a portable terminal device that is provided with a function that manages biological sound information that is acquired using the stethoscope 30, and a function that indicates an observation sequence to the operator U by displaying an indication screen to the operator U. Additionally, for example, it is possible to include a tablet terminal, a smartphone, a personal digital assistant (PDA), a notebook computer or the like as examples of a portable terminal device, but a portable terminal device is not to these examples. In addition, a portable terminal device is preferable from a viewpoint of usability, but naturally, the terminal device 100 may also be a desktop type personal computer.

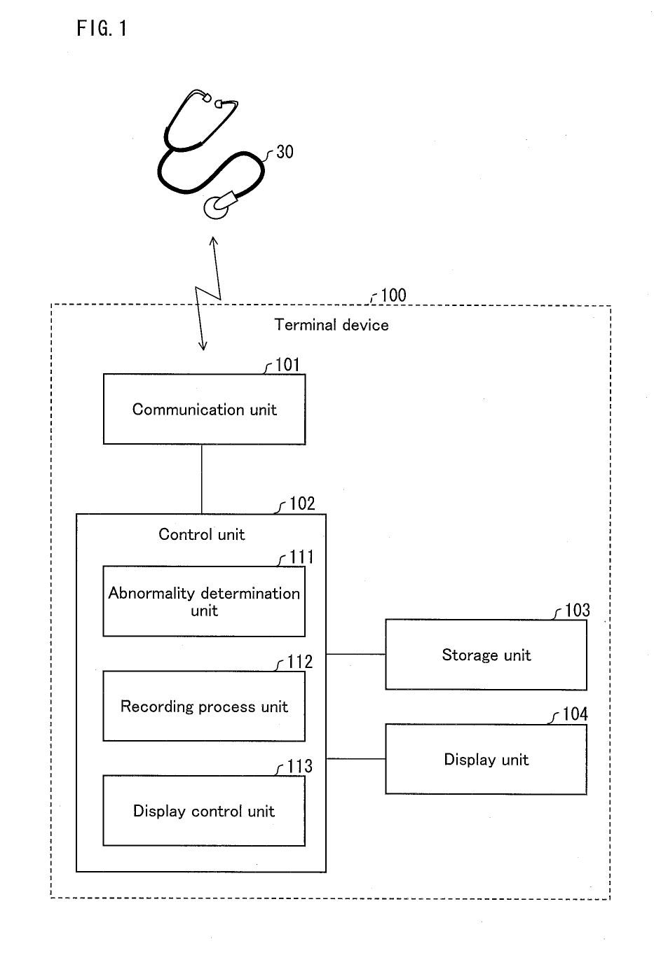

As shown in FIG. 1, the terminal device 100 is provided with a communication unit 101, a control unit 102, a storage unit 103 and a display unit 104.

The communication unit 101 is a block that performs the transmission and receipt of information with the stethoscope 30, and in addition, performs the transmission and receipt of information with the server device 40 through the network 50 (FIG. 2). In particular, the communication unit 101 receives biological sound information from the stethoscope 30.

The storage unit 103 is a unit on which control programs that the control unit 102 executes, an OS program, an application program and various kinds of data that are read when these programs are executed, are stored. The storage unit 103 is configured by a non-volatile memory device such as a hard disk or flash memory. In addition, the storage unit 103 stores biological sound information that the communication unit 101 receives and various kinds of information that is linked to biological sound information.

The display unit 104 is for example, a display device such as a liquid crystal display, and displays an electronic health record of a patient P, screens for managing biological sound information, an indication screen for indicating an observation sequence to the operator U of the stethoscope 30 and the like. In addition, the display unit 104 of the present embodiment has a touch panel function, and a user can operate the terminal device 100 using the touch panel function.

(Control Unit 102)

The control unit 102 is a computer that performs control of the terminal device 100. In particular, the control unit 102 is configured to perform a process that displays an indication screen 150 on the display unit 104 in the manner that is shown in FIGS. 5A to 5F, a process that determines whether or not biological sound information during auscultation is an abnormality candidate (a sound that has a high probability of being an abnormal sound), and a process for recording on the storage unit 103 by associating abnormality information that shows whether or not the corresponding biological sound information is an abnormality candidate with observation position information (the observation number) that shows the observation position of the corresponding biological sound information.

As shown in FIG. 1, the control unit 102 is provided with an abnormality determination unit 111, a recording process unit 112 and a display control unit 113.

(Abnormality Determination Unit 111)

The abnormality determination unit 111 is a block into which biological sound information that is sent from the stethoscope 30 to the communication unit 101 is input, and which performs a determination process that determines whether or not the biological sound information is an abnormality candidate (a sound that has a high probability of being an abnormal sound).

The abnormality determination unit 111 determines whether or not the biological sound information is an abnormality candidate using a well-known method. It is possible to include a method that performs the determination by comparing the biological sound information with sample data as the well-known method. For example, it is possible to include a process in which a plurality of kinds of abnormal state sample data are stored in the storage unit 103, the abnormality determination unit 111 calculates a degree of similarity between input biological sound information and each item of sample data by using a well-known waveform matching technique, the biological sound information is determined to be an abnormality candidate if sample data for which the degree of similarity is greater than or equal to a threshold value is present, and the biological sound information is determined as normal (not an abnormality candidate) if such sample data is not present, as one example. Additionally, although there are features which differ from the corresponding process, it is possible to include PTL 3 to PTL 5 as examples of techniques that determine whether or not biological sound information is abnormal (an abnormality candidate) by comparing the biological sound information with sample data.

Alternatively, the abnormality determination unit 111 may be configured to calculate characteristic quantities that are derived from a waveform of the biological sound information without using sample data in the above-described manner, and determine whether or not the biological sound information is an abnormality candidate from the characteristic quantities. A method that performed this kind of determination is for example, shown in Japanese Patent Application No. 2011-272785 (which was yet to be published at the time of the priority date of the present application), which is a prior application by the applicant of the present application. The determination that is used in this method will be described below.

The abnormality determination unit 111 calculates an autocorrelation function of a waveform of biological sound information, a two-dimensional spectrum that is obtained by performing Fourier transformation of a waveform of biological sound information, and a three-dimensional spectrogram that is obtained by performing Fourier transformation of a waveform of biological sound information every predetermined time interval as characteristic quantities. Additionally, the two-dimensional spectrum has frequency and frequency components as the axes thereof, and three-dimensional spectrogram has frequency, frequency components and time as the axes thereof.

Next, the abnormality determination unit 111 performs the following processes A to C.

Process A: The abnormality determination unit 111 outputs true in a case where a waveform of the autocorrelation function has a peak at a period of 2 to 5 seconds, and a peak width at ¼ of an amplitude value of the peak of an envelope curve of the autocorrelation function is less than or equal to 10% of a respiratory cycle (a period of one respiration), and outputs false otherwise.

Process B: The abnormality determination unit 111 outputs true if a sum of frequency components of less than or equal to 200 Hz in the two-dimensional spectrum is greater than or equal to 80% of a total of all frequency components, and outputs false otherwise.

Process C: The abnormality determination unit 111 outputs true if there is periodicity in frequency components of greater than or equal to 400 Hz in the three-dimensional spectrogram, and outputs false otherwise. Additionally, there is periodicity if it is found that a timing with which signal components that are greater than or equal to a given amount are generated, is an interval of a predetermined number of seconds (for example, 3 seconds), and there is not periodicity is the timing is not an interval of the predetermined number of seconds.

The abnormality determination unit 111 is configured to determine that biological sound information is normal if all of the outputs of processes A to C are true, and determines that biological sound information is an abnormality candidate if at least one of the outputs of processes A to C is false.

In any case, as long as the determination process by the abnormality determination unit 111 is a determination process that outputs a determination result that shows whether or not biological sound information input from the stethoscope 30 is an abnormality candidate (a sound for which there is a possibly of showing an abnormality), any method may be applied, and the contents of the process are not limited.

Further, the abnormality determination unit 111 transmits abnormality sound information that shows a determination result to the recording process unit 112 and the display control unit 113. The abnormality sound information is information that shows whether or not biological sound information is an abnormality candidate.

(Recording Process Unit 112)

When a record button 500, which is shown on an indication screen (refer to FIG. 5) that is displayed on the display unit 104, is pressed, the recording process unit 112 is configured to input a predetermined amount of time of biological sound information from the communication unit 101 as biological sound information for which a recording instruction has been input from the operator U, and store the input biological sound information in the storage unit 103.

However, rather than merely recording the biological sound information in the storage unit 103, the recording process unit 112 records by linking an observation number (positional information) that shows an observation position (a designated observation position) that is pointed out as a position which should be observed at the current point in time, abnormality sound information, which is a determination result (the most recent determination result) of the abnormality determination unit 111, and the biological sound information. Additionally, the observation position (a designated observation position) that is pointed out as a position which should be observed at the current point in time refers to an observation position that is pointed out as a position which should be observed at the current point in time on the indication screen 150 that is displayed on the display unit 104 at the current point in time (refer to reference numeral 400 in FIG. 5).

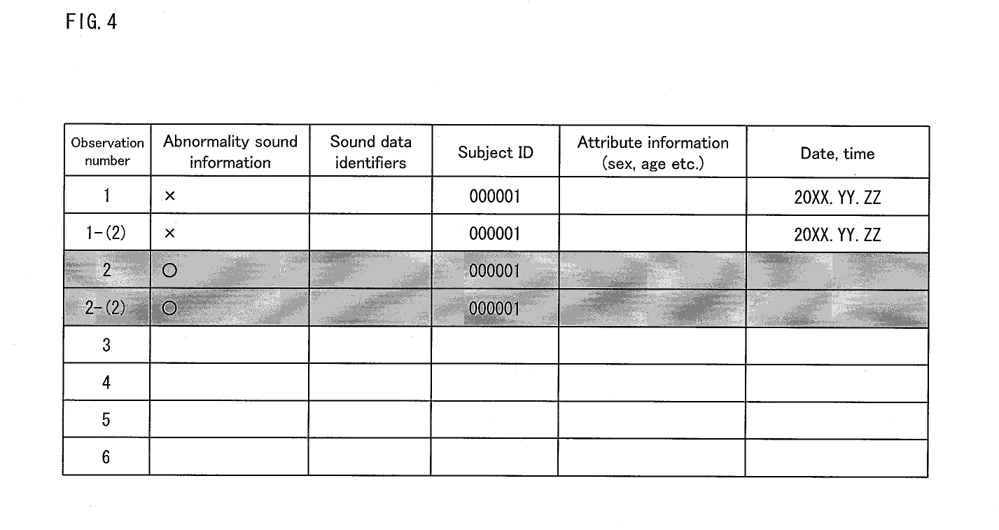

For example, the recording process unit 112 stores biological sound information, which is a recording target, in the storage unit 103, and creates and stores an observation table, in which identifiers (file numbers or the like) of the observation number, the abnormality sound information, and the biological sound information are associated, in the storage unit 103. FIG. 4 is an example of an observation table that is stored in a storage unit 103. Abnormality sound information, which is shown with either a symbol X or a symbol O is shown in the example that is shown in FIG. 4. A symbol X shows that the biological sound information is normal, and a symbol O shows that the biological sound information an abnormality candidate. In addition, the “sound data identifiers” in FIG. 4 correspond to identifiers of the biological sound information. Furthermore, in the manner of the observation table that is shown in FIG. 4, a subject ID (an ID of a patient), attribute information of the subject, a date and time of auscultation and the like may also be recorded in association with the identifiers.

(Display Control Unit 113)

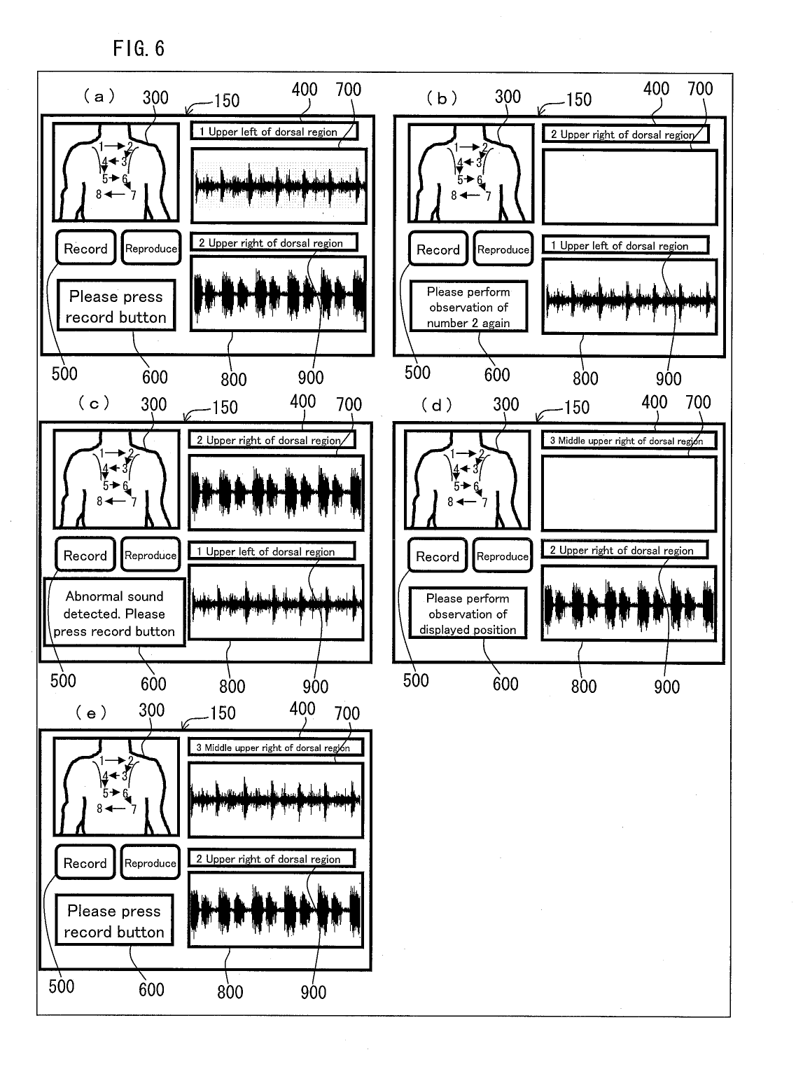

The display control unit 113 is a block that performs display control of the display unit 104. In addition, in the present embodiment, by displaying the indication screens 150 that are shown in FIG. 5 and FIG. 6 on the display unit 104, the display control unit 113 is configured to designate (indicate) an observation sequence of observation (auscultation) using the auscultation system 200 to the operator U. Hereinafter, observation will be described using the indication screens 150.

In the auscultation system 200, information that shows observation positions and the observation sequence of each observation position is stored in the storage unit 103 in advance, and the display control unit 113 is configured to set an observation number that shows the observation position and the observation sequence (the order) of the corresponding observation position on the basis of the above-described information. For example, in a case where a dorsal region mode, which is a mode that observes the dorsal region, is selected by the operator U, the display control unit 113 sets the observation numbers 1 to 8 that are shown in FIG. 3(a). The observation numbers are information that specify the observation positions and a basic observation sequence (the order) of the observation positions. In other words, the positions that are attached to each observation number in FIG. 3(a) are observation positions that show each observation number, and the numeral of each observation number refers to a basic observation sequence. That is, fundamentally, observation is configured to be performed in order from observation positions with smaller observation numbers.

Additionally, FIG. 3(a) is a diagram that shows observation numbers in a case where the dorsal region mode is selected, and in a case where a precordial region mode, which is a mode that observes the precordial region, is selected by the operator U, rather than FIG. 3(a), the display control unit 113 is configured to set the observation numbers 1 to 8 that are shown in FIG. 3(b). However, in the following text, for the sake of convenience, description will be given based on the assumption that the dorsal region mode is selected by the operator U.

When the operator U inputs an initiation command for the dorsal region mode to the terminal device 100 by operating the terminal device 100, the display control unit 113 sets the observation numbers 1 to 8 in the manner that is shown in FIG. 3(a). Further, the display control unit 113 displays the indication screen 150 that is shown in FIG. 5(a) on the display unit 104. As shown in FIG. 5(a), a pseudo human body image 300 on which each observation number is attached to an observation position that corresponds to each observation number, an observation position box 400 that shows an observation position which should be observed at the current point in time (also referred to as a “designated observation position”), the record button 500, an indication box 600 in which message text for indicating operation is shown, a received waveform box 700 that shows a waveform of biological sound information that the terminal device 100 is receiving, a recording waveform box 800 that shows a waveform of recorded biological sound information, and a recorded observation position box 900 that shows an observation position that corresponds to biological sound information that is being shown in the recording waveform box 800, are shown on the indication screen 150.

The display control unit 113 conveys a designated observation position to an operator by showing a designated observation position, which is an observation position which should be observed at the current point in time, in the observation position box 400 of the indication screen 150. Additionally, as shown in FIG. 5(a), in the present embodiment, the observation number of a designated observation position and text that shows a name of the corresponding designated observation position are shown in the observation position box 400. In addition, since the observation number of a designated observation position that is shown first is 1, as shown in FIG. 5(a), an observation initiation time, is shown by a “1” in the observation position box 400.

Further, the operator U performs observation according to the indication (text) that is shown in the indication box 600 of the indication screen 150 of FIG. 5(a). In other words, the operator U auscultates a position of an observation number 1 in a state in which the indication screen 150 in FIG. 5(a) is being displayed.

When the operator U initiates auscultation, the communication unit 101 receives biological sound information from the stethoscope 30. When the communication unit 101 receives biological sound information, as shown in FIG. 5(b), the display control unit 113 displays a waveform of the biological sound information that is being received in the received waveform box 700, and displays a message that designates the operator U to press the record button 500 in the indication box 600.

Additionally, when the communication unit 101 receives biological sound information, the abnormality determination unit 111 performs a determination of whether or not a corresponding biological sound signal is an abnormality candidate, but in this instance, it is assumed that the abnormality determination unit 111 has determined that the biological sound information that is being received (the biological sound information of the observation position of the observation number 1) is normal (not an abnormality candidate).

Further, when the operator U presses the record button 500, the recording process unit 112 performs the recording of biological sound information. When recording is performed, as shown in FIG. 5(c), the display control unit 113 ends the waveform display of the received waveform box 700, and in place of this, displays a waveform of biological sound information that is recorded in the recording waveform box 800, and displays an observation position that corresponds to the biological sound information that was recorded in the recorded observation position box 900.

Thereafter, when the operator U completes auscultation of the observation position that corresponds to the observation number 1, the communication unit 101 also ends the reception of the biological sound signal that corresponds to the observation number 1.

In the above-described manner, when the recording of the biological sound signal that corresponds to the observation number 1 is completed, and the reception of the biological sound signal that corresponds to the observation number 1 also ends, the display control unit 113 displays the observation number of a designated observation position in the observation position box 400 with an observation position which should be observed after the observation number 1 as the designated observation position.

In this instance, in the present embodiment, if, in the first observation of an observation position A, it is determined by the abnormality determination unit 111 that the biological sound signal is normal, an observation number of an observation position B which should be observed after the observation position A becomes a number that is 1 plus the observation number of the observation position A.

Therefore, in the above-described description, since it is determined by the abnormality determination unit 111 that the biological sound information of the observation position of the observation number 1 is normal, the observation number of an observation position that is to be observed after the observation position of the observation number 1, is 2. Accordingly, when the recording of the biological sound signal that corresponds to the observation number 1 is completed, and the reception of the biological sound signal that corresponds to the observation number 1 also ends, as shown in FIG. 5(d), the display control unit 113 displays the observation position of an observation number 2 in the observation position box 400 as the designated observation position. As a result of this, by designating the observation number 2 to the operator U as the designated observation position, the operator U repeats the same operation as that of the observation number 1 for the observation number 2.

Accordingly, supposing that the biological sound signal of each observation position is continuously determined to be normal by the abnormality determination unit 111, the system is configured so that indication and operation such as that described above are repeated, and recording of each observation position is performed once in an order from the observation number 1 to the observation number 8.

(Case in which Abnormality Candidate is Determined)

Incidentally, in the present embodiment, when, in the first observation of a given observation position, it is determined by the abnormality determination unit 111 that the biological sound signal is an abnormality candidate, the display control unit 113 is configured to perform indication of the designated observation position so that observation is performed twice for each of the corresponding observation position, and an observation position that has a bilaterally symmetrical relationship with the corresponding observation position (hereinafter referred to as a “symmetrical position”). Firstly, the reason for this will be described below.

In a case where a doctor performs diagnosis of a given observation position, rather than diagnosis being performed from biological sounds of the corresponding observation position only, diagnosis is performed by taking a comparison result with biological sounds of a symmetrical position into consideration. This is because, (a) even if the biological sounds of a given observation position are unusual, there are case in which these sounds correspond to normal sounds for the patient P who is an auscultation target, and therefore, it is not sufficient to only use biological sounds of the observation position as an absolute assessment, and (b) judging from the fact that the thickness and shape of organs, or the like which have a bilaterally symmetrical positional relationship in a patient P who is an auscultation target, are similar to one another, since the biological sounds of an observation position and a symmetrical position should be similar to one another if the observation position and the symmetrical position are normal, a comparison of the biological sounds of the observation position and the biological sounds of the symmetrical position is effective in an accurate diagnosis.

Further, in a case where a doctor listens to biological sounds that are detected from a given observation position and thinks that there is a suspicion of an abnormality, rather than concluding diagnosis with a single auscultation of each of the corresponding observation position and the symmetrical position, there is a tendency to improve the precision of the diagnosis through repeated auscultation. This is because, in a single auscultation, there is also a chance that there are circumstances in which the biological sounds deviate from normal sounds or the like due to mere noise or the like.

In contrast to this, in the auscultation system 200 of the present embodiment, the operator U may be a person other than a doctor, and therefore, it is possible to assume a format in which a doctor performs diagnosis using recorded data. For this reason, even if auscultation is performed in this kind of format, in a case where there is biological sound information that is determined to be an abnormality candidate by a computer, if auscultation is performed and recorded twice for each of an observation position that corresponds to the corresponding biological sound information and the symmetrical position of the observation position, it is possible to improve the subsequent precision of the diagnosis by a doctor.

Next, the symmetrical position will be described. In the present embodiment, as shown in FIGS. 3(a) and 3(b), the observation positions of observation numbers 1 to 8 are set. In this instance, in a case where a straight line that extends from the top to the bottom of the human body and bisects the left side and the right side thereof, is assumed, the symmetrical position refers to a position that is in axial symmetry with the observation position with the straight line as an axis thereof. If described more specifically, a symmetrical position of an arbitrary observation position refers to (a) an observation position that is different from the above-described arbitrary observation position, and (b) in a case where a straight line that extends from the top to the bottom of the human body and bisects the left side and the right side thereof, is assumed, an observation position that is in axial symmetry with the above-described arbitrary observation position with the straight line as an axis thereof.

That is, it can be said that the symmetrical position is a position that has a paired relationship with an observation position according to a predetermined rule, which is the above-described axially symmetrical relationship with the corresponding observation position (in other words, it can be said that, according to a predetermined rule in which a position that is axially symmetrical to an arbitrary observation position is called a symmetrical position, the symmetrical position is a position that has a paired relationship with the arbitrary observation position).

For this reason, in FIGS. 3(a) and 3(b), the observation position of the observation number 1 and the observation position of the observation number 2 have a paired relationship, the observation position of the observation number 1 is in a position which is symmetrical to the observation position of the observation number 2, and the observation position of the observation number 2 is in a position which is symmetrical to the observation position of the observation number 1. In the same manner, the observation position of the observation number 3 and the observation position of the observation number 4 have a paired relationship, the observation position of the observation number 3 is in a position which is symmetrical to the observation position of the observation number 4, and the observation position of the observation number 4 is in a position which is symmetrical to the observation position of the observation number 3. In addition, in the same manner, the observation position of the observation number 5 and the observation position of the observation number 6 have a paired relationship, the observation position of the observation number 5 is in a position which is symmetrical to the observation position of the observation number 6, and the observation position of the observation number 6 is in a position which is symmetrical to the observation position of the observation number 5. Furthermore, in the same manner, the observation position of the observation number 7 and the observation position of the observation number 8 have a paired relationship, the observation position of the observation number 7 is in a position which is symmetrical to the observation position of the observation number 8, and the observation position of the observation number 8 is in a position which is symmetrical to the observation position of the observation number 7. In other words, the observation positions of odd-numbered observation numbers have a bilaterally symmetrical paired relationship with the observation positions of even-numbered observation numbers which are 1 plus the number of the corresponding even number.

Next, the indication process of the display control unit 113 in a case where it is determined by the abnormality determination unit 111 in the first observation of a given observation position that a biological sound signal an abnormality candidate, will be described.

(Case in which Biological Sound Signal of Observation Position of Even-Numbered Observation Number is Abnormality Candidate)

Firstly, a case in which biological sound information that is obtained by the first observation of an observation position of an even-numbered observation number is determined to be an abnormality candidate by the abnormality determination unit 111 will be described.

In a case where an observation number of an observation position that is being observed (is being auscultated) is an even number, the observation number of the symmetrical position of the observation position that is being observed is an odd number that is one minus the observation number of the observation position that is being observed. For this reason, in a case where an observation position with an even observation number is being observed (is being auscultated), and the observation of the corresponding observation position is the first observation thereof, the observation of a symmetrical position of the observation position that is being observed has already been completed. Therefore, when the observation of an observation position with an even observation number is completed, observation has been completed once for each of the corresponding observation position and the symmetrical position of the corresponding observation position.

In such an instance, in a case where the biological sound information is determined by the abnormality determination unit 111 to be an abnormality candidate in the first observation of an observation position with an even observation number, the display control unit 113 performs indication so that re-observation is performed once for each of the corresponding observation position and the symmetrical position after the observation of the corresponding observation position has been completed (or in other words, after recording has been completed).

In other words, in a case where biological sound information that is obtained in the first observation of an observation position of an even-numbered observation number a is an abnormality candidate, after the observation has been completed, the display control unit 113 makes the operator U perform re-observation of an observation position of an odd-numbered observation number b that is one minus the observation number a by indicating an observation sequence with the observation position of the observation number b as the designated observation position. That is, by displaying the observation position of the observation number b in the observation position box 400 of the indication screen 150, and displaying an indication that designates the operator U to press the record button 500 in the indication box 600, the display control unit 113 makes the operator U perform re-observation of the observation position of the observation number b (biological sound information of the observation position of the observation number b is recorded).

Thereafter, regardless of whether or not it is determined that the biological sound information that is obtained in performing re-observation of the observation position of the observation number b is an abnormality candidate, by indicating an observation sequence with the observation position of the observation number a as the designated observation position, the display control unit 113 makes the operator U perform re-observation of the observation position of the observation number a. That is, by displaying the observation position of the observation number a in the observation position box 400 of the indication screen 150, and displaying an indication that designates the operator U to press the record button 500 in the indication box 600, the display control unit 113 makes the operator U perform re-observation of the observation position of the observation number a (biological sound information of the observation position of the observation number a is recorded).

As a result of this, observation is carried out twice for each of the observation position of the even-numbered observation number a and the observation position (the symmetrical position) of the odd-numbered observation number b that is one minus the observation number a. Subsequently, by indicating an observation sequence with an observation position of an observation number c that is 1 plus the observation number a as the designated observation position, the display control unit 113 makes the operator U perform a first observation for the observation position of the observation number c.

Next, an example of an indication screen that is displayed in a case where biological sound information is determined to be an abnormality candidate by the abnormality determination unit 111 in the first observation of an observation position with an even observation number will be described on the basis of FIGS. 5 and 6.

As described using FIGS. 5(a) to 5(c), it is determined that the biological sound information for the observation position of the observation number 1 is normal, and the observation is completed, and as shown in FIG. 5(d), the observation number 2 is shown in the observation position box 400, and the indication of an observation sequence for the observation number 2 is initiated. The operator U initiates auscultation of the observation position of the observation number 2, the communication unit 101 receives the biological sound information of the observation position of the observation number 2, and the abnormality determination unit 111 performs determination of whether or not the corresponding biological sound information is an abnormality candidate. In this instance, it is assumed that the abnormality determination unit 111 has determined that the biological sound information of the observation position of the observation number 2 is an abnormality candidate. In this case, as shown in FIG. 5(e), the display control unit 113 displays the waveform of the biological sound information of the observation number 1 that was recorded most recently in the recording waveform box 800, and displays a message to the effect that an abnormal sound has been detected, and a message that designates pressing of the record button 500 in the indication box 600 while displaying the waveform of the biological sound information of the observation number 2 that is being received in the received waveform box 700.

Further, when the operator U presses the record button 500, the recording process unit 112 performs recording of the biological sound information that is being received. When recording is performed, as shown in FIG. 5(f), the display control unit 113 ends the waveform display of the received waveform box 700, displays the waveform of the biological sound information of the observation number 2 that was recorded most recently in the recording waveform box 800, and displays an observation position that corresponds to the biological sound information that was recorded in the recorded observation position box 900. In addition, as shown in FIG. 5(f), by displaying the observation position of the observation number 1, which is the symmetrical position of the observation position of the observation number 2, in the observation position box 400, the display control unit 113 designates (indicates) the operator U to perform re-observation of the observation position of the observation number 1.

Further, the operator U initiates re-auscultation of the observation position of the observation number 1 according to the indication screen 150 of FIG. 5(f), the communication unit 101 receives the biological sound information of the observation position of the observation number 1, and the abnormality determination unit 111 performs determination of whether or not the corresponding biological sound information is an abnormality candidate. Subsequently, as shown in FIG. 6(a), the display control unit 113 displays the waveform of the biological sound information of the observation number 2 that was recorded most recently in the recording waveform box 800, and displays a message that designates pressing of the record button 500 in the indication box 600 while displaying the waveform of the biological sound information of the observation number 1 that is being received in the received waveform box 700.

When the operator U presses the record button 500 of the indication screen 150 that is shown in FIG. 6(a), the recording process unit 112 performs recording of the biological sound information that is being received (the biological sound information of the observation position of the observation number 1). When recording is performed, as shown in FIG. 6(b), the display control unit 113 ends the waveform display of the received waveform box 700, displays the waveform of the biological sound information of the observation number 1 that was recorded most recently in the recording waveform box 800, and displays an observation position that corresponds to the biological sound information that was recorded in the recorded observation position box 900.

Additionally, the biological sound information of the observation number 1 that is recorded in this instance is biological sound information that is obtained by performing re-observation, but due to the fact that the observation number 1 is already stored in the table that is shown in FIG. 4 since the first observation of the observation number 1 has already been performed, the biological sound information of the observation number 1 of the first observation overlaps with that of the observation number 1 of the re-observation. In such an instance, in the present embodiment, for re-observations, the recording process unit 112 is configured to store a number to which a hyphen and a (2) have been added to the original observation number as a new number in the table of FIG. 4. Therefore, as shown in FIG. 4, an observation number 1 which is linked to the biological sound information that is obtained in the first observation, and an observation number 1-(2) which is linked to the biological sound information that is obtained in the re-observation are stored for the observation position of the observation number 1.

In addition, when re-observation (recording) of the observation position of observation number 1 is completed in the above-described manner, as shown in FIG. 6(b), by displaying the observation position of the observation number 2 in the observation position box 400, the display control unit 113 designates (indicates) the operator U to perform re-observation of the observation position of the observation number 2. Further, as shown in FIGS. 6(c) and 6(d), recording is also performed in the same manner as that of the observation number 1 for the observation number 2 as a result of the record button 500 being pressed, and re-observation is completed.

In this manner, in a case where it is determined, during the first observation of the even-numbered observation number 2, that the biological sound signal thereof is an abnormality candidate, re-observation of the observation position of the observation number 1 and re-observation of the observation position of the observation number 2 are performed, and as a result of this, observation is performed twice for each of the observation position of the observation number 1 and the observation position of the observation number 2.

Subsequently, as shown in FIG. 6(d), by displaying the observation position of the observation number 3 in the observation position box 400, the display control unit 113 designates (indicates) the operator U to perform a first observation of the observation position of the observation number 3. Further, when the operator U initiates auscultation of the observation position of the observation number 3, as shown in FIG. 6(e), the display control unit 113 displays the waveform of the biological sound signal that is obtained from the observation position of the observation number 3 in the received waveform box 700, and displays a message that designates pressing of the record button 500 in the indication box 600. In this manner, observation is performed in the same manner as that of the observation positions up to observation number 2 for observation positions of observation number 3 and upwards.

(Case in which Biological Sound Signal of Observation Position of Odd-Numbered Observation Number is Abnormality Candidate)

Next, a case in which it is determined by the abnormality determination unit 111 that the biological sound information that is obtained by the first observation of the observation position with an odd observation number is an abnormality candidate will be described.

In a case where an observation number of an observation position that is being observed (is being auscultated) is an odd number, the observation number of the symmetrical position of the observation position that is being observed is an even number that is one plus the observation number of the observation position that is being observed. For this reason, in a case where an observation position with an odd observation number is being observed (is being auscultated), and the observation of the corresponding observation position is the first observation thereof, the observation of a symmetrical position of the observation position that is being observed has not been completed. Therefore, when the first observation of an observation position with an odd observation number is completed, observation has been completed once for the corresponding observation position, but observation has not been performed for the symmetrical position of the corresponding observation position.

In such an instance, in a case where the biological sound information is determined by the abnormality determination unit 111 to be an abnormality candidate in the first observation of an observation position with an odd observation number, the display control unit 113 performs indication so that re-observation is performed for the corresponding observation position, and a first observation and re-observation are performed for the symmetrical position after the observation of the corresponding observation position has been completed (or in other words, after recording has been completed).

More specifically, in a case where biological sound information that is obtained in the first observation of an observation position of an odd-numbered observation number e is an abnormality candidate, after the observation has been completed, the display control unit 113 makes the operator U perform a first observation of an observation position of an even-numbered observation number f that is one plus the observation number e by indicating an observation sequence with the observation position of the observation number f as the designated observation position. That is, by displaying the observation position of the observation number f in the observation position box 400 of the indication screen 150, and displaying an indication that designates the operator U to press the record button 500 in the indication box 600, the display control unit 113 makes the operator U perform observation of the observation position of the observation number f (biological sound information of the observation position of the observation number f is recorded).

Thereafter, regardless of whether or not it is determined that the biological sound information that is obtained in performing observation of the observation position of the observation number f is an abnormality candidate, by indicating an observation sequence with the observation position of the observation number e as the designated observation position, the display control unit 113 makes the operator U perform re-observation of the observation position of the observation number e. That is, by displaying the observation position of the observation number e in the observation position box 400 of the indication screen 150, and displaying an indication that designates the operator U to press the record button 500 in the indication box 600, the display control unit 113 makes the operator U perform re-observation of the observation position of the observation number e (biological sound information of the observation position of the observation number e is recorded).

Furthermore, regardless of whether or not it is determined that the biological sound information that is obtained in performing re-observation of the observation position of the observation number e is an abnormality candidate, by indicating an observation sequence with the observation position of the observation number f as the designated observation position, the display control unit 113 makes the operator U perform re-observation of the observation position of the observation number f. That is, by displaying the observation position of the observation number f in the observation position box 400 of the indication screen 150, and displaying an indication that designates the operator U to press the record button 500 in the indication box 600, the display control unit 113 makes the operator U perform re-observation of the observation position of the observation number f (biological sound information of the observation position of the observation number e is recorded).

As a result of this, observation is carried out twice for each of the observation position of the odd-numbered observation number e and the observation position of the even-numbered observation number f that is one plus the observation number e. Subsequently, regardless of whether or not it is determined that the biological sound information that is obtained in performing re-observation of the observation position of the observation number f is an abnormality candidate, by indicating an observation sequence with an observation position of an observation number g that is 1 plus the observation number f as the designated observation position, the display control unit 113 makes the operator U perform a first observation for the observation position of the observation number g.

(Process Flow in Auscultation System 200)

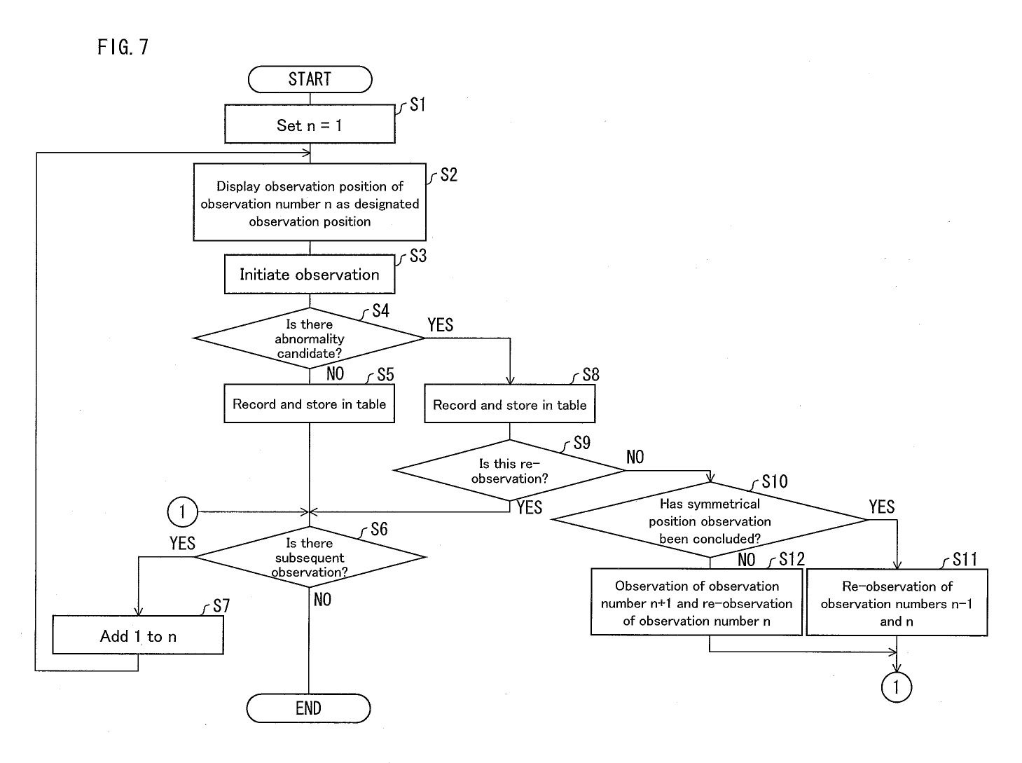

Next, an example of a process flow in the auscultation system 200 will be described with reference to FIG. 7. FIG. 7 is a flowchart that shows an example of a process flow in the auscultation system 200.

When the operator U inputs an execution instruction for the dorsal region mode into the terminal device 100, the control unit 102 is configured to set n=1 (S1), and display the observation position of an observation number n as the designated observation position in the observation position box 400 of the indication screen 150 (S2).

Further, the operator U initiates auscultation (observation) of the observation position of the observation number n according to the indication of the indication box 600 of the indication screen 150 (S3). When auscultation is initiated, the control unit 102 determines whether or not the biological sound information that is obtained by the auscultation is an abnormality candidate (S4).

In this instance, in a case where it is determined that the biological sound information is not an abnormality candidate (NO in S4), or in other words, in a case where the biological sound information is normal, the control unit 102 records the biological sound information when the operator U presses the record button 500 of the indication screen 150, and stores a sound data identifier, the observation number, and the abnormality sound information of the biological sound information in an observation table (S5). Additionally, in S5, since the biological sound information is normal, abnormality sound information of symbol X (normal) is configured to be stored in the observation table in association with the observation number (positional information), the audio data identifier, and the like (refer to FIG. 4).

After S5, the control unit 102 determines whether or not there is still an observation position that should be observed next (S6). That is, there is not an observation position that should be observed next if n is 8 and there is an observation position that should be observed next if n is less than or equal to 7.

Therefore, in a case where n=1, the control unit 102 is configured to determine that there is still an observation position that should be observed next (YES in S6), add 1 to n (S7), and repeat the processes from S2 onwards. Further, in a case where the processes from S2 onwards are repeated and the process moves to the S6 after n has become 8, the control unit 102 is configured to determine that there is not still an observation position that should be observed next (NO in S6), and complete the process.

Next, a process after the control unit 102 has determined that the biological sound information is an abnormality candidate in S4 will be described.

In a case where it is determined that the biological sound information is an abnormality candidate (YES in S4), in the same manner as that of S5, the control unit 102 records the biological sound information when the operator U presses the record button 500 of the indication screen 150, and stores a sound data identifier, the observation number, and the abnormality sound information of the biological sound information in an observation table (S8). Additionally, in S8, since the biological sound information is an abnormality candidate, abnormality sound information of symbol O (abnormal) is configured to be stored in the observation table in association with the observation number (observation position information), the audio data identifier, and the like (refer to FIG. 4).

Next, the control unit 102 determines whether or not the processes that were performed in S3, S4 and S8 correspond to re-observation (S9). In a case where the processes correspond to re-observation (YES in S9), the control unit 102 moves the process to S6.

In contrast to this, in a case where the processes that were performed in S3, S4 and S8 do not correspond to re-observation (NO in S9), the control unit 102 determines whether or not observation of a symmetrical position with respect to the observation position of the observation number n has been concluded (S10). That is, observation of the symmetrical position has been concluded if n is an even number, and observation of the symmetrical position has not been concluded if n is an odd number.

In a case where observation of the symmetrical position with respect to the observation position of the observation number n has been concluded (YES in S10), re-observation of the observation position of an observation number n−1 and re-observation of the observation position of the observation number n are performed (S11). In other words, in S11, the same processes as those of S2, S3, S4 and S5 (or S8) are performed with the observation position of the observation number n−1 as the designated observation position, and thereafter, the same processes as those of S2, S3, S4 and S5 (or S8) are performed with the observation position of the observation number n as the designated observation position.

In addition, in a case where it is determined, in S10, that observation of the symmetrical position with respect to the observation position of the observation number n has not been concluded (NO in S10), observation of an observation position of the observation number n+1, and re-observation of the observation position of the observation number n are performed (S12). In other words, in S12, the same processes as those of S2, S3, S4 and S5 (or S8) are performed with the observation position of the observation number n+1 as the designated observation position, and thereafter, the same processes as those of S2, S3, S4 and S5 (or S8) are performed with the observation position of the observation number n as the designated observation position.

After S11 or S12 have been completed, the process moves to S6, and the control unit 102 determines whether or not there is still an observation position that should be observed next. In a case where there is still an observation position that should be observed next (YES in S6), the control unit 102 adds 1 to n (S7), and repeats the processes from S2 onwards. In a case where there is not still an observation position that should be observed next (NO in S6), the control unit 102 is configured to complete the process.

In the above-described flowchart, in a case where for example, n=2 (an even number), and it is determined that the biological sound information is an abnormality candidate in the first observation of the observation position of the observation number 2 (YES in S4), the first observation of the observation position of the observation number 2 is finished by recording the biological sound information of the observation position of the observation number 2 in S8. Thereafter, NO is selected in S9, YES is selected in S10, and in S11, re-observation of the observation position of the observation number 2 and re-observation of the observation position of the observation number 1 are performed. Subsequently, the process passes S6, n becomes 3 in S7, and the process moves to the processes of the observation number 3. In this manner, in a case where it is determined that the biological sound information of the observation position of the observation number 2 (an even number) is an abnormality candidate in the first observation, observation is performed twice for each of the observation position of the observation number 2 and the observation number 1, which is the symmetrical position of the observation number 2.

In addition, in the above-described flowchart, in a case where n=1 (an odd number), and it is determined that the biological sound information is an abnormality candidate in the first observation of the observation position of the observation number 1 (YES in S4), the first observation of the observation position of the observation number 1 is finished by recording the biological sound information of the observation position of the observation number 1 in S8. Thereafter, NO is selected in S9, NO is selected in S10, and in S12, a first observation of the observation position of the observation number 2 and re-observation of the observation position of the observation number 1 are performed. Subsequently, the process passes S6, and n becomes 2 in S7. Furthermore, the re-observation of the observation position of the observation number 2 is subsequently performed by the process passing S2, S3, S4 and S5 (or S8). Further, the process moves to S6 in a case where the process has passed S5, and the process moves to S6 after YES has been selected in S9 in a case where the process has passed S8. After the process has moved to S6, n becomes 3 in S7, and the process moves to the processes of the observation number 3. In this manner, in a case where it is determined that the biological sound information of the observation position of the observation number 1 (an odd number) is an abnormality candidate in the first observation, observation is performed twice for each of the observation position of the observation number 1 and the observation number 2, which is the symmetrical position of the observation number 1.

In the manner described above, the terminal device 100 of the present embodiment is provided with the abnormality determination unit 111, the recording process unit 112, and the display control unit 113. By displaying the indication screen 150, the display control unit 113 indicates observation positions on which the stethoscope 30 is to be placed to the operator U (user) of the stethoscope 30. The recording process unit 112 performs a biological sound observation process, which is a process for recording on the storage unit (memory device) 103 by associating an observation number (positional information) that shows an observation position that is indicated by the display control unit 113 with biological sound information that is being input from the stethoscope 30. The abnormality determination unit 111 determines whether or not the biological sound information is an abnormality candidate. Further, in a case where it is determined by the abnormality determination unit 111 that the biological sound information is an abnormality candidate, the display control unit 113 performs the indication so that the biological sound observation process is executed at least twice for each of an observation position that is shown by a positional number that is associated with the biological sound information that was determined to be an abnormality candidate (an abnormality observation position), and a symmetrical position of the corresponding observation position (a predetermined observation position).

As a result of this, in a case where biological sound information for which there is a suspicion of an abnormality, is acquired (a case in which the biological sound information is determined to be an abnormality candidate), the biological sound observation process is performed at least twice for each of the observation position, which is an acquisition source of the corresponding biological sound information, and the symmetrical position of the corresponding observation position. Therefore, since a doctor, who subsequently performs diagnosis by reproducing the biological sound information, has the opportunity to listen to at least two items of biological sound information for each of the observation position for which there is a suspicion of an abnormality and the symmetrical position of the corresponding observation position, there is an effect of being capable of improving the precision of the diagnosis.

In addition, in the present embodiment, the abnormality determination unit 111 is configured to output abnormality sound information that indicates whether or not the biological sound information is an abnormality candidate, and the recording process unit 112 is configured to record in the storage unit 103 by associating the positional number with the biological sound information and the abnormality sound information. Further, since the server device 40 in FIG. 2 can access the storage unit 103 of the terminal device 100, a doctor or the like who operates the server device 40 can reproduce the biological sound information of the storage unit 103, and it is possible to display and confirm the positional number and the abnormality sound information that are associated with the biological sound information. For this reason, since it is possible to alert a doctor or the like who attempts to reproduce the biological sound information of an abnormality candidate that there is a possibility of an abnormality in advance, the doctor or the like listens to the biological sound information carefully, and therefore, there is an effect of being capable of improving the precision of the diagnosis.

In addition, in the indication screens 150 of FIG. 5, the display control unit 113 displays an image in which all of the observation positions (observation positions for which instruction (indication) as a position that should be observed has not yet been performed and observation positions for which the above-described instruction has been concluded) are shown on the pseudo human body image 300 as observation numbers, but the display control unit 113 may make the display form of observation numbers of observation positions (abnormality observation positions) that correspond to biological sound information that is determined to be an abnormality candidate, and the display form of observation numbers of observation positions other than abnormality observation positions differ. For example, the display forms may be made different by making the colors thereof different, or the display forms may be made different by the presence or absence of a flashing light. As a result of this, from the point of view of the operator U, an effect of being able to know, among all of the observation positions for which auscultation was performed, observation positions for which there is a possibility of an abnormality, at a glance, is exhibited.

Furthermore, in the images (FIG. 5) in which all of the observation positions are shown as observation numbers on the pseudo human body image 300, the display control unit 113 may make the display form of observation numbers of observation positions (abnormality observation positions) that correspond to biological sound information that is determined to be an abnormality candidate, and the display form of observation numbers of symmetrical positions (predetermined observation positions) of the corresponding abnormality observation positions, and the display form of observation positions other than the abnormality observation positions and the predetermined observation positions differ. In other words, the display form of the abnormality observation positions and the display form of the symmetrical positions of the abnormality observation position are set as the same, but the display form of the abnormality observation position and the symmetrical position of the abnormality observation positions and the display form of observation positions which are not abnormality observation positions, and are not symmetrical positions of abnormality observation positions are made to differ (additionally, the display forms of a plurality of abnormality observation positions are set to be the same as each other). As a result of this, from the point of view of the operator U, an effect of being able to know, among all of the observation positions for which auscultation was performed, pairs of observation positions for which re-observation was performed at a glance, is exhibited.

### Embodiment 2

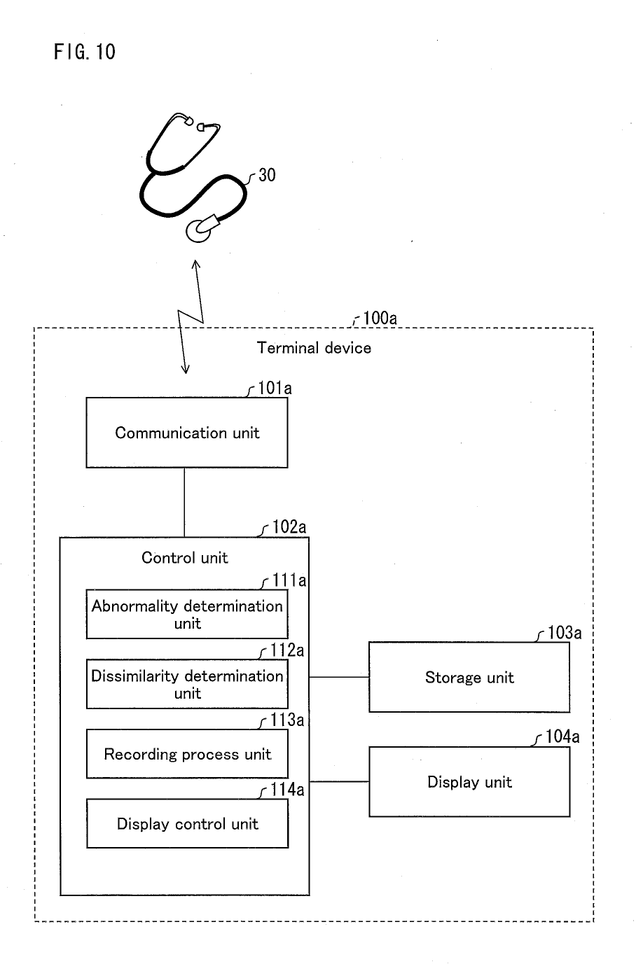

Next, Embodiment 2 will be described. Embodiment 2 is an embodiment in which a terminal device 100a (refer to FIG. 10) is used in place of the terminal device 100 in the auscultation system 200 in FIG. 2. Hereinafter, Embodiment 2 will be described in detail. Additionally, since the outline of the auscultation system 200 in FIG. 2 and the configuration of the stethoscope 30 are as described in Embodiment 1, the description thereof have been omitted in this instance.

(Configuration of Terminal Device 100a)

In the same manner as the terminal device 100, the terminal device 100a is a portable terminal device that is provided with a function that manages biological sound information that is acquired using the stethoscope 30, and a function that indicates an observation sequence to the operator U by displaying an indication screen to the operator U. Additionally, for example, it is possible to include a tablet terminal, a smartphone, a personal digital assistant (PDA), a notebook computer or the like as examples of a portable terminal device, but a portable terminal device is not to these examples. In addition, a portable terminal device is preferable from a viewpoint of usability, but naturally, the terminal device 100a may also be a desktop type personal computer.

As shown in FIG. 10, the terminal device 100a is provided with a communication unit 101a, a control unit 102a, a storage unit 103a and a display unit 104a.

Since the communication unit 101a, the storage unit 103a and the display unit 104a have the same configurations as those of the communication unit 101, the storage unit 103 and the display unit 104 that were described in Embodiment 1, the description thereof have been omitted.

(Control Unit 102a)

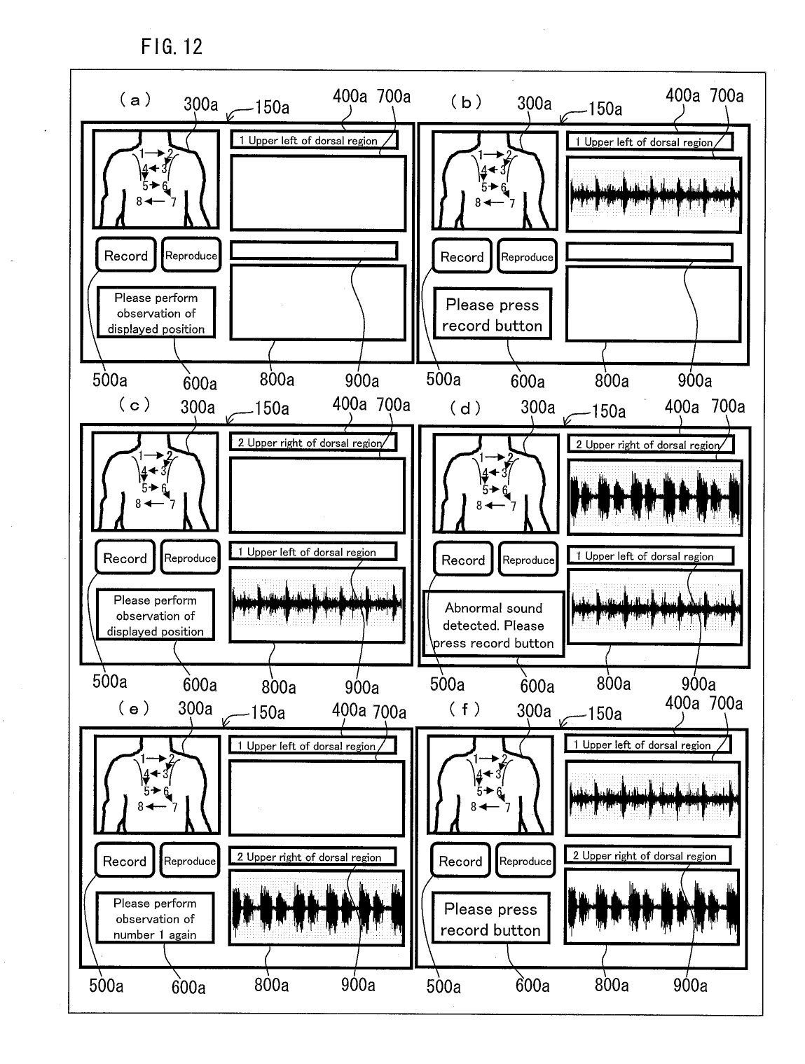

The control unit 102a is a computer that performs control of the terminal device 100a. In particular, the control unit 102a is configured to perform a process that displays an indication screen 150a on the display unit 104a in the manner that is shown in FIGS. 12(a) to 12(f), a process that determines whether or not biological sound information during auscultation is an abnormality candidate (a sound that has a high probability of being an abnormal sound), and a process for recording on the storage unit 103a by associating abnormality information that shows whether or not the corresponding biological sound information is an abnormality candidate with observation position information (the observation number) that shows the observation position of the corresponding biological sound information.

As shown in FIG. 10, the control unit 102a is provided with an abnormality determination unit 111a, a dissimilarity determination unit 114a, a recording process unit 112a and a display control unit 113a.

(Abnormality Determination Unit 111a)

Since the configuration of the abnormality determination unit 111a is the same as that of the abnormality determination unit 111 that was described in Embodiment 1, and the content of the determination process that the abnormality determination unit 111a executes is also the same, the descriptions thereof have been omitted. However, the abnormality determination unit 111a of the present embodiment transmits abnormality sound information that shows a determination result to the dissimilarity determination unit 114a, the recording process unit 112a and the display control unit 113a. The abnormality sound information is information that shows whether or not biological sound information is an abnormality candidate.

(Dissimilarity Determination Unit 114a)

In the present embodiment, in a case where the abnormality determination unit 111a determines that biological sound information that is obtained in the first observation of a given observation position is normal, and the abnormality determination unit 111a determines that biological sound information that is obtained in the first observation of a symmetrical position of the corresponding observation position is normal, the dissimilarity determination unit 114a is configured to determine whether or not the biological sound information of the corresponding observation position and the biological sound information of the corresponding symmetrical position are not similar. The reason for this will be described below (additionally, the meaning of symmetrical position is the same as that of Embodiment 1).

In a case where a doctor performs diagnosis of a given observation position, rather than diagnosis being performed from biological sounds of the corresponding observation position only, diagnosis is performed by taking a comparison result with biological sounds of a symmetrical position into consideration. This is because, (a) even if the biological sounds of a given observation position are unusual, there are case in which these sounds correspond to normal sounds for the patient P who is an auscultation target, and therefore, it is not sufficient to only use biological sounds of the observation position as an absolute assessment, and (b) judging from the fact that the thickness and shape of organs, or the like which have a bilaterally symmetrical positional relationship in a patient P who is an auscultation target, are similar to one another, since the biological sounds of an observation position and a symmetrical position should be similar to one another if the observation position and the symmetrical position are normal, a comparison of the biological sounds of the observation position and the biological sounds of the symmetrical position is effective in an accurate diagnosis.

That is, if the observation position and the symmetrical position are normal, the biological sounds of the observation position and the biological sounds of the symmetrical position tend to be similar to one another, and if there is a suspicion of an abnormality for at least one of the observation position and the symmetrical position, the biological sounds of the observation position and the biological sounds of the symmetrical position tend not to be similar to one another.

In such an instance, in the auscultation system 200 of the present embodiment, in a case where both the biological sound information of the first observation of a given observation position, and the biological sound information of the first observation of the symmetrical position of the corresponding observation position are determined to be normal by the abnormality determination unit 111a, it is determined by the dissimilarity determination unit 114a whether or not the biological sound information of the corresponding observation position and the biological sound information of the corresponding symmetrical position are not similar, and the system is configured to store the determination result thereof.

Next, the content of the determination process of the dissimilarity determination unit 114a will be described. The dissimilarity determination unit 114a is configured to detect pairs of a given observation position and a symmetrical position of the corresponding observation position that are both determined to be normal as a determination target on the basis of the abnormality sound information that is sent from the abnormality determination unit 111a.

Further, the dissimilarity determination unit 114a determines whether or not the biological sound information of the observation position that is a determination target and the biological sound information of the symmetrical position that is a determination target are not similar.

The determination method in this instance can use a well-known determination method that uses waveform pattern matching. In other words, it is possible to include a method that compares a predetermined characteristic quantity that is derived from a waveform of one set of biological sound information with a predetermined characteristic quantity that is derived from a waveform of another set of biological sound information, determines that the sets of biological sound information are not similar if a difference in the characteristic quantities is greater than or equal to a threshold value, and determines that the sets of biological sound information are not dissimilar if a difference in the characteristic quantities is less than a threshold value.

A specific example of the determination method will be described in detail below. The dissimilarity determination unit 114a outputs a two-dimensional spectrum of the waveform of the biological sound information of an observation position that is a determination target and a two-dimensional spectrum of the waveform of the biological sound information of the symmetrical position by performing Fourier transformation of the waveform of the biological and the waveform of the biological sound information of the symmetrical position. The two-dimensional spectrum is a spectrum that has frequency as a horizontal axis thereof and frequency components (signal intensity) as a vertical axis thereof, and three-dimensional spectrogram has frequency, frequency components and time as the axes thereof.

Next, it is possible to include a determination method in which the dissimilarity determination unit 114a is configured to calculate a sum of frequency components of a predetermined frequency band (for example, less than or equal to 200 Hz) as a first characteristic quantity for each of the spectra that are output, determine that the spectra are not similar if the first characteristic quantity that is obtained from the biological sound information of the observation position is outside a range of 90% to 110% of the first characteristic quantity that is obtained from the biological sound information of the symmetrical position, and determine that the spectra are similar if the first characteristic quantity that is obtained from the biological sound information of the observation position is within a range of 90% to 110% of the first characteristic quantity that is obtained from the biological sound information of the symmetrical position, as an example of a determination method.

In addition, a determination method in which the dissimilarity determination unit 114a is configured to calculate a signal intensity of a predetermined frequency as a second characteristic quantity for each of the spectra that are output, determine that the spectra are not similar if the second characteristic quantity that is obtained from the biological sound information of the observation position is outside a range of 90% to 110% of the second characteristic quantity that is obtained from the biological sound information of the symmetrical position, and determine that the spectra are similar if the second characteristic quantity that is obtained from the biological sound information of the observation position is within a range of 90% to 110% of the second characteristic quantity that is obtained from the biological sound information of the symmetrical position, may also be used.

Alternatively, the dissimilarity determination unit 114a may be configured to determine that the spectra are similar in a case where the first characteristic quantity that is obtained from the biological sound information of the observation position is outside a range of 90% to 110% of the first characteristic quantity that is obtained from the biological sound information of the symmetrical position, and the second characteristic quantity that is obtained from the biological sound information of the observation position is outside a range of 90% to 110% of the second characteristic quantity that is obtained from the biological sound information of the symmetrical position, and determine that the spectra are similar in other cases.

Additionally, the characteristic quantities that are used in the above-described determination are not limited to the sum of frequency components of a predetermined frequency band (for example, less than or equal to 200 Hz) and the signal intensity of a predetermined frequency, and any kind of parameter may be used as long as the parameter can be derived from the waveform of the biological sound information using a well-known characteristic quantity extraction method, and is a characteristic quantity that can be used in waveform matching.

In any case, as long as the determination process by the dissimilarity determination unit 114a compares a characteristic quantity that is extracted from a waveform of biological sound information of a given observation position and a characteristic quantity that is extracted from a waveform of biological sound information of the symmetrical position of the corresponding observation position, and is waveform pattern matching that determines whether or not both waveforms are not similar on the basis of the comparison result, any well-known method may be applied, and the contents of the process are not limited.

The dissimilarity determination unit 114a transmits dissimilarity determination information that shows a determination result of whether or not the waveform of the biological sound information of a given observation position and the waveform of the biological sound information of the symmetrical position of the corresponding observation position are not similar to the recording process unit 112a and the display control unit 113a.

(Recording Process Unit 112a)

When the record button 500a, which is shown on an indication screen (refer to FIG. 12) that is displayed on the display unit 104a, is pressed, the recording process unit 112a is configured to input a predetermined amount of time of biological sound information from the communication unit 101a as biological sound information for which a recording instruction has been input from the operator U, and store the input biological sound information in the storage unit 103a.

However, rather than merely recording the biological sound information in the storage unit 103a, the recording process unit 112a records by linking an observation number that shows an observation position, abnormality sound information, which is a determination result (the most recent determination result) of the abnormality determination unit 111a, the biological sound information and dissimilarity determination information, which is a determination result of the dissimilarity determination unit 114a.

For example, the recording process unit 112a stores biological sound information, which is a recording target, in the storage unit 103a, and creates and stores an observation table, in which identifiers (file numbers or the like) of the observation number, the abnormality sound information, the dissimilarity determination information and the biological sound information are associated, in the 103a.

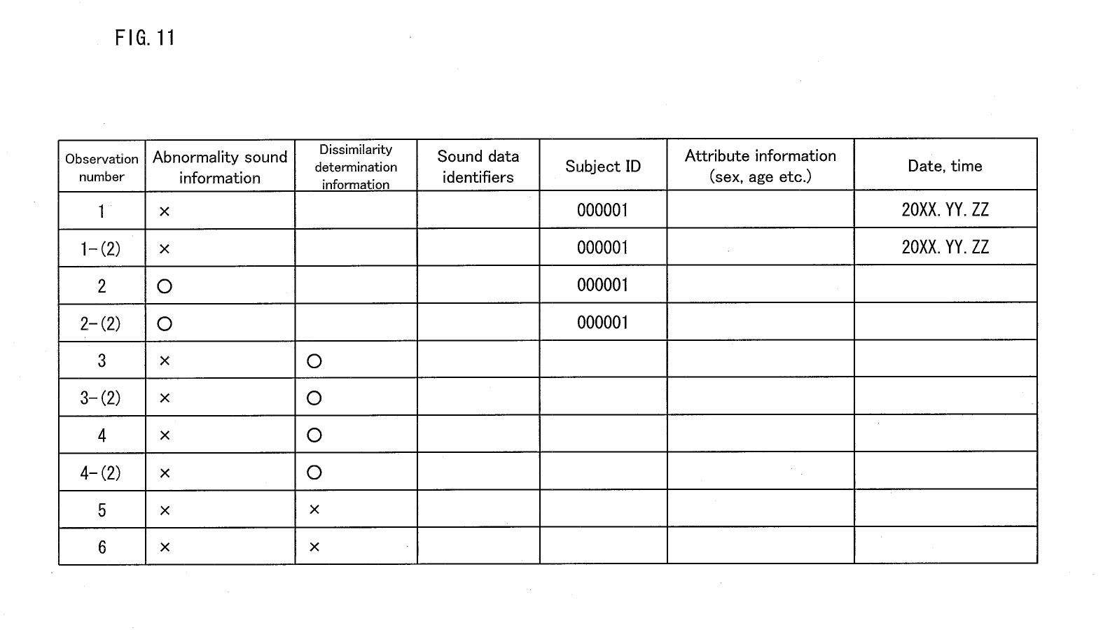

FIG. 11 is an example of an observation table that is stored in a storage unit 103a. Abnormality sound information, which is shown with either a symbol X or a symbol O is shown in the example that is shown in FIG. 11. A symbol X for abnormality sound information shows that the biological sound information is a normal sound, and a symbol O for abnormality sound information shows that the biological sound information is an abnormal sound. In addition, dissimilarity information, which is shown with either a symbol X or a symbol O is shown in the example that is shown in FIG. 11. A symbol X for dissimilarity information shows that the sets of biological sound information are similar, and a symbol O for dissimilarity information shows that the sets of biological sound information are not similar. In addition, the “sound data identifiers” in FIG. 11 correspond to identifiers of the biological sound information. Furthermore, in the manner of the observation table that is shown in FIG. 11, a subject ID (an ID of a patient), attribute information of the subject, a date and time of auscultation and the like may also be recorded in association with the identifiers.

(Display Control Unit 113a)

The display control unit 113a is a block that performs display control of the display unit 104a. In addition, in the present embodiment, by displaying the indication screens 150a that are shown in FIG. 12 on the display unit 104a, the display control unit 113a is configured to designate (indicate) an observation sequence of observation (auscultation) using the auscultation system 200 to the operator U. Hereinafter, observation will be described using the indication screens 150a.

In the auscultation system 200, information that shows observation positions and the observation sequence of each observation position is stored in the storage unit 103a in advance, and the display control unit 113a is configured to set an observation number that shows the observation position and the observation sequence (the order) of the corresponding observation position on the basis of the above-described information. For example, in a case where a dorsal region mode, which is a mode that observes the dorsal region, is selected by the operator U, the display control unit 113a sets the observation numbers 1 to 8 that are shown in FIG. 3(a). The observation numbers are information that specify the observation positions and a basic observation sequence (the order) of the observation positions. In other words, the positions that are attached to each observation number in FIG. 3(a) are observation positions that show each observation number, and the numeral of each observation number refers to a basic observation sequence. That is, fundamentally, observation is configured to be performed in order from observation positions with smaller observation numbers.

Additionally, FIG. 3(a) is a diagram that shows observation numbers in a case where the dorsal region mode is selected, and in a case where a precordial region mode, which is a mode that observes the precordial region, is selected by the operator U, rather than FIG. 3(a), the display control unit 113a is configured to set the observation numbers 1 to 8 that are shown in FIG. 3(b). However, in the following text, for the sake of convenience, description will be given based on the assumption that the dorsal region mode is selected by the operator U.

When the operator U inputs an initiation command for the dorsal region mode to the terminal device 100a by operating the terminal device 100a, the display control unit 113a sets the observation numbers 1 to 8 in the manner that is shown in FIG. 3(a). Further, the display control unit 113a displays the indication screen 150a that is shown in FIG. 12A on the display unit 104a. As shown in FIG. 12(a), a pseudo human body image 300a on which each observation number is attached to an observation position that corresponds to each observation number, an observation position box 400a that shows an observation position that is pointed out by the display control unit 113a as a position which should be observed at the current point in time (also referred to as a “designated observation position”), the record button 500a, an indication box 600a in which message text for indicating operation is shown, a received waveform box 700a that shows a waveform of biological sound information that the terminal device 100a is receiving, a recording waveform box 800a that shows a waveform of recorded biological sound information, and a recorded observation position box 900a that shows an observation position that corresponds to biological sound information that is being shown in the recording waveform box 800a, are shown on the indication screen 150a.

The display control unit 113a conveys a designated observation position to an operator by showing a designated observation position, which is an observation position which should be observed at the current point in time, in the observation position box 400a of the indication screen 150a. Additionally, as shown in FIG. 12(a), in the present embodiment, the observation number of a designated observation position and text that shows a name of the corresponding designated observation position are shown in the observation position box 400a. In addition, since the observation number of a designated observation position that is shown first is 1, as shown in FIG. 12(a), an observation initiation time, is shown by a “1” in the observation position box 400a.

Further, the operator U performs observation according to the indication (text) that is shown in the indication box 600a of the indication screen 150a of FIG. 12(a). In other words, the operator U auscultates a position of an observation number 1 in a state in which the indication screen 150a in FIG. 12(a) is being displayed.

When the operator U initiates auscultation, the communication unit 101a receives biological sound information from the stethoscope 30. When the communication unit 101a receives biological sound information, as shown in FIG. 12(b), the display control unit 113a displays a waveform of the biological sound information that is being received in the received waveform box 700a, and displays a message that designates the operator U to press the record button 500a in the indication box 600a.

Additionally, when the communication unit 101a receives biological sound information, the abnormality determination unit 111a performs a determination of whether or not a corresponding biological sound signal is an abnormality candidate, but in this instance, it is assumed that the abnormality determination unit 111a has determined that the biological sound information that is being received (the biological sound information of the observation position of the observation number 1) is normal (not an abnormality candidate).

Further, when the operator U presses the record button 500a, the recording process unit 112a performs the recording of biological sound information. When recording is performed, as shown in FIG. 12(c), the display control unit 113a ends the waveform display of the received waveform box 700a, and in place of this, displays a waveform of biological sound information that is recorded in the recording waveform box 800a, and displays an observation position that corresponds to the biological sound information that was recorded in the recorded observation position box 900a.

Thereafter, when the operator U completes auscultation of the observation position that corresponds to the observation number 1, the communication unit 101a also ends the reception of the biological sound signal that corresponds to the observation number 1.

In the above-described manner, when the recording of the biological sound signal that corresponds to the observation number 1 is completed, and the reception of the biological sound signal that corresponds to the observation number 1 also ends, the display control unit 113a displays the observation number of a designated observation position in the observation position box 400a with an observation position which should be observed after the observation number 1 as the designated observation position.

In this instance, if, in the first observation of a given observation position (assuming a case of an observation position A), it is determined by the abnormality determination unit 111a that the biological sound signal is normal, fundamentally, an observation number of an observation position which should be observed after the observation position A becomes a number that is 1 plus the observation number of the observation position A.

Therefore, in the above-described description, since it is determined by the abnormality determination unit 111a that the biological sound information of the observation position of the observation number 1 is normal, the observation number of an observation position that is to be observed after the observation position of the observation number 1, is 2. Accordingly, when the recording of the biological sound signal that corresponds to the observation number 1 is completed, as shown in FIG. 12(c), the display control unit 113a displays the observation position of an observation number 2 in the observation position box 400a as the designated observation position. As a result of this, the observation number 2 is designated to the operator U as the designated observation position, and the operator U repeats the same operation as that of the observation number 1 for the observation number 2.

(Case in which Abnormality Candidate is Determined)

When, in the first observation of a given observation position, it is determined by the abnormality determination unit 111a that the biological sound signal is an abnormality candidate, the display control unit 113a is configured to perform indication of the designated observation position so that observation is performed twice for each of the corresponding observation position, and an observation position (the symmetrical position) that has a bilaterally symmetrical relationship with the corresponding observation position. The reason for this will be described below.

The fact that diagnosis is performed by taking a comparison result with biological sounds of a symmetrical position into consideration rather than diagnosis being performed from biological sounds of the corresponding observation position only in a case where a doctor performs diagnosis of a given observation position, is as described above. Further, in a case where a doctor listens to biological sounds that are detected from a given observation position and thinks that there is a suspicion of an abnormality, rather than concluding diagnosis with a single auscultation of each of the corresponding observation position and the symmetrical position, there is a tendency for the precision of the diagnosis to be improved by performing re-auscultation (re-observation) (in other words, observation is performed twice for each of the corresponding observation position and the symmetrical position). This is because, in a single auscultation, there is also a chance that there are circumstances in which the biological sounds deviate from normal sounds or the like due to mere noise or the like. In contrast to this, in the auscultation system 200 of the present embodiment, the operator U may be a person other than a doctor, and therefore, it is possible to assume a format in which a doctor performs diagnosis using recorded data. For this reason, even if auscultation is performed in this kind of format, in a case where there is biological sound information that is determined to be an abnormality candidate by a computer, if re-observation is performed and recorded for each of an observation position that corresponds to the corresponding biological sound information and the symmetrical position of the observation position (auscultation is performed and recorded twice), it is possible to improve the subsequent precision of the diagnosis by a doctor.

Next, the indication process of the display control unit 113a in a case where it is determined by the abnormality determination unit 111a in the first observation of a given observation position that a biological sound signal an abnormality candidate, will be described.

(Case in which Biological Sound Signal of Observation Position of Even-Numbered Observation Number is Abnormality Candidate)

Firstly, a case in which biological sound information that is obtained by the first observation of an observation position of an even-numbered observation number is determined to be an abnormality candidate by the abnormality determination unit 111a will be described.

In a case where an observation number of an observation position that is being observed (is being auscultated) is an even number, the observation number of the symmetrical position of the observation position that is being observed is an odd number that is one minus the observation number of the observation position that is being observed. For this reason, in a case where an observation position with an even observation number is being observed (is being auscultated), and the observation of the corresponding observation position is the first observation thereof, the observation of a symmetrical position of the observation position that is being observed has already been completed. Therefore, when the observation of an observation position with an even observation number is completed, observation has been completed once for each of the corresponding observation position and the symmetrical position of the corresponding observation position.

In such an instance, in a case where the biological sound information is determined by the abnormality determination unit 111a to be an abnormality candidate in the first observation of an observation position with an even observation number, the display control unit 113a performs indication so that re-observation is performed once for each of the corresponding observation position and the symmetrical position after the observation of the corresponding observation position has been completed (or in other words, after recording has been completed).

In other words, in a case where biological sound information that is obtained in the first observation of an observation position of an even-numbered observation number (assuming a case of an observation number a) is an abnormality candidate, after the observation has been completed, the display control unit 113a makes the operator U perform re-observation of an observation position of an odd-numbered observation number (assuming a case of an observation number b) that is one minus the observation number a by indicating an observation sequence with the observation position of the observation number b as the designated observation position. That is, by displaying the observation position of the observation number b in the observation position box 400a of the indication screen 150, and displaying an indication that designates the operator U to press the record button 500a in the indication box 600a, the display control unit 113a makes the operator U perform re-observation of the observation position of the observation number b (biological sound information of the observation position of the observation number b is recorded).

Thereafter, regardless of whether or not it is determined that the biological sound information that is obtained in performing re-observation of the observation position of the observation number b is an abnormality candidate, by indicating an observation sequence with the observation position of the observation number a as the designated observation position, the display control unit 113a makes the operator U perform re-observation of the observation position of the observation number a. That is, by displaying the observation position of the observation number a in the observation position box 400a of the indication screen 150a, and displaying an indication that designates the operator U to press the record button 500 in the indication box 600a, the display control unit 113a makes the operator U perform re-observation of the observation position of the observation number a (biological sound information of the observation position of the observation number a is recorded).

As a result of this, observation is carried out twice for each of the observation position of the even-numbered observation number a and the observation position (the symmetrical position) of the odd-numbered observation number b that is one minus the observation number a. Subsequently, by indicating an observation sequence with an observation position of an observation number that is 1 plus the observation number a (assuming a case of an observation number c) as the designated observation position, the display control unit 113a makes the operator U perform a first observation for the observation position of the observation number c.

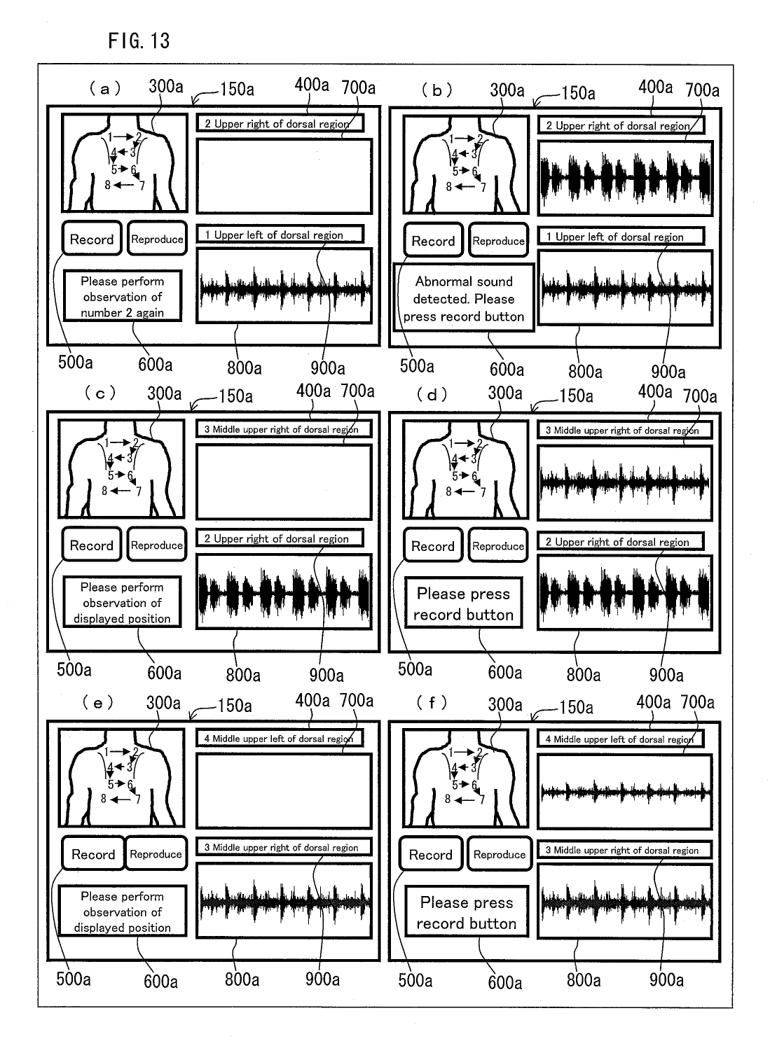

Next, an example of an indication screen that is displayed in a case where biological sound information is determined to be an abnormality candidate by the abnormality determination unit 111a in the first observation of an observation position with an even observation number will be described on the basis of FIGS. 12 and 13.

As described using FIGS. 12(a), 12(b), and 12(c), it is determined that the biological sound information for the observation position of the observation number 1 is normal, and the observation is completed, and as shown in FIG. 12(c), the observation number 2 is shown in the observation position box 400a, and the indication of an observation instruction for the observation number 2 is initiated. The operator U initiates auscultation of the observation position of the observation number 2 according to the indication.

When the operator U initiates auscultation, the communication unit 101a receives the biological sound information of the observation number 2. In this instance, it is assumed that the abnormality determination unit 111a has determined that the biological sound information of the observation position of the observation number 2 is an abnormality candidate. In this case, as shown in FIG. 12(d), the display control unit 113a displays the waveform of the biological sound information of the observation number 2 that is being received in the received waveform box 700a, and displays a message to the effect that an abnormal sound has been detected, and a message that designates pressing of the record button 500a in the indication box 600a.

Further, when the operator U presses the record button 500a, the recording process unit 112a performs recording of the biological sound information that is being received (or in other words, the biological sound information of the observation number 2). When recording is performed, as shown in FIG. 12(e), the display control unit 113a ends the waveform display of the received waveform box 700a, displays the waveform of the biological sound information of the observation number 2 that was recorded most recently in the recording waveform box 800a, and displays an observation position that corresponds to the biological sound information that was recorded in the recorded observation position box 900a. In addition, as shown in FIG. 12(e), by displaying the observation position of the observation number 1, which is the symmetrical position of the observation position of the observation number 2, in the observation position box 400a, and displaying a message of a re-observation instruction for the observation number 1 in the indication box 600a, the display control unit 113a designates (indicates) the operator U to perform re-observation of the observation position of the observation number 1.

Further, the operator U initiates re-auscultation of the observation position of the observation number 1 according to the indication screen 150a of FIG. 12(e). As a result of this, the communication unit 101a receives the biological sound information of the observation position of the observation number 1, and the abnormality determination unit 111a performs determination of whether or not the corresponding biological sound information is an abnormality candidate. Further, as shown in FIG. 12(f), the display control unit 113a displays a message that designates pressing of the record button 500a in the indication box 600a while displaying the waveform of the biological sound information of the observation number 1 that is being received in the received waveform box 700a.

When the operator U presses the record button 500a of the indication screen 150a that is shown in FIG. 12(f), the recording process unit 112a performs recording of the biological sound information that is being received (the biological sound information of the observation position of the observation number 1). When recording is performed, as shown in FIG. 13(a), the display control unit 113a ends the waveform display of the received waveform box 700a, displays the waveform of the biological sound information of the observation number 1 that was recorded most recently in the recording waveform box 800a, and displays an observation position that corresponds to the biological sound information that was recorded in the recorded observation position box 900a.

Additionally, the biological sound information of the observation number 1 that is recorded in this instance is biological sound information that is obtained by performing re-observation, but due to the fact that the observation number 1 is already stored in the table that is shown in FIG. 11 since the first observation of the observation number 1 has already been performed, the biological sound information of the observation number 1 of the first observation overlaps with that of the observation number 1 of the re-observation. In such an instance, in the present embodiment, for re-observations, the recording process unit 112a is configured to store a number to which “-(2)” has been added to the observation number in the table of FIG. 11. Therefore, as shown in FIG. 11, for the observation position of the observation number 1, “1” is linked to the biological sound information that is obtained in the first observation, and “1-(2)” is linked to the biological sound information that is obtained in the re-observation.

In addition, when re-observation (recording) of the observation position of observation number 1 is completed in the above-described manner, as shown in FIG. 13(a), by displaying the observation position of the observation number 2 in the observation position box 400a, and displaying a message of a re-observation instruction of the observation number 2 in the indication box 600a, the display control unit 113a designates (indicates) the operator U to perform re-observation of the observation position of the observation number 2. Further, as shown in FIGS. 13(b) and 13(c), recording is also performed in the same manner as that of the observation number 1 for the observation number 2 as a result of the record button 500a being pressed, and re-observation is completed.

In this manner, in a case where it is determined, during the first observation of the even-numbered observation number 2, that the biological sound signal thereof is an abnormality candidate, re-observation of the observation position of the observation number 1 and re-observation of the observation position of the observation number 2 are performed, and as a result of this, observation is performed a total of two times for each of the observation position of the observation number 1 and the observation position of the observation number 2.

Subsequently, as shown in FIG. 13(c), by displaying the observation position of the observation number 3 in the observation position box 400a, the display control unit 113a designates (indicates) the operator U to perform a first observation of the observation position of the observation number 3. Further, when the operator U initiates auscultation of the observation position of the observation number 3, as shown in FIG. 13(d), the display control unit 113a displays the waveform of the biological sound signal that is obtained from the observation position of the observation number 3 in the received waveform box 700a, and displays a message that designates pressing of the record button 500a in the indication box 600a. In this manner, observation is performed in the same manner as that of the observation positions up to observation number 2 for the observation position of observation number 3.

(Case in which Biological Sound Signal of Observation Position of Odd-Numbered Observation Number is Abnormality Candidate)

Next, a case in which it is determined by the abnormality determination unit 111a that the biological sound information that is obtained by the first observation of an observation position with an odd observation number is an abnormality candidate will be described.

In a case where an observation number of an observation position that is being observed (is being auscultated) is an odd number, the observation number of the symmetrical position of the observation position that is being observed is an even number that is one plus the observation number of the observation position that is being observed. For this reason, in a case where an observation position with an odd observation number is being observed (is being auscultated), and the observation of the corresponding observation position is the first observation thereof, the observation of a symmetrical position of the observation position that is being observed has not been completed. Therefore, when the first observation of an observation position with an odd observation number is completed, observation has been completed once for the corresponding observation position, but observation has not been performed for the symmetrical position of the corresponding observation position.

In such an instance, in a case where the biological sound information is determined by the abnormality determination unit 111a to be an abnormality candidate in the first observation of an observation position with an odd observation number, the display control unit 113a performs indication so that re-observation is performed for the corresponding observation position, and a first observation and re-observation are performed for the symmetrical position after the observation of the corresponding observation position has been completed (or in other words, after recording has been completed).

In other words, in a case where biological sound information that is obtained in the first observation of an observation position of an odd-numbered observation number (assuming a case of an observation number e) is an abnormality candidate, after the observation has been completed, the display control unit 113a makes the operator U perform a first observation of an observation position of an even-numbered observation number (assuming a case of an observation number f) that is one plus the observation number e by indicating an observation sequence with the observation position of the observation number f as the designated observation position. That is, by displaying the observation position of the observation number f in the observation position box 400a of the indication screen 150a, and displaying an indication that designates the operator U to press the record button 500a in the indication box 600a, the display control unit 113a makes the operator U perform observation of the observation position of the observation number f (biological sound information of the observation position of the observation number f is recorded).

Thereafter, regardless of whether or not it is determined that the biological sound information that is obtained in performing observation of the observation position of the observation number f is an abnormality candidate, by indicating an observation sequence with the observation position of the observation number e as the designated observation position, the display control unit 113a makes the operator U perform re-observation of the observation position of the observation number e. That is, by displaying the observation position of the observation number e in the observation position box 400a of the indication screen 150a, and displaying an indication that designates the operator U to press the record button 500a in the indication box 600a, the display control unit 113a makes the operator U perform re-observation of the observation position of the observation number e (biological sound information of the observation position of the observation number e is recorded).

Furthermore, regardless of whether or not it is determined that the biological sound information that is obtained in performing re-observation of the observation position of the observation number e is an abnormality candidate, by indicating an observation sequence with the observation position of the observation number f as the designated observation position, the display control unit 113a makes the operator U perform re-observation of the observation position of the observation number f. That is, by displaying the observation position of the observation number f in the observation position box 400a of the indication screen 150a, and displaying an indication that designates the operator U to press the record button 500a in the indication box 600a, the display control unit 113a makes the operator U perform re-observation of the observation position of the observation number f (biological sound information of the observation position of the observation number e is recorded).

As a result of this, a first observation and re-observation are performed (observation is carried out a total of two times) for each of the observation position of the odd-numbered observation number e and the observation position of the even-numbered observation number f that is one plus the observation number e. Subsequently, regardless of whether or not it is determined that the biological sound information that is obtained in performing re-observation of the observation position of the observation number f is an abnormality candidate, by indicating an observation sequence with an observation position of an observation number g that is 1 plus the observation number f as the designated observation position, the display control unit 113a makes the operator U perform a first observation for the observation position of the observation number g.

(Case in which Dissimilarity Determination Unit 114a Determines Dissimilarity)

In addition, in the present embodiment, in a case where the abnormality determination unit 111a determines that a biological sound signal of a first observation of a given observation position, and determines that a biological sound signal of a first observation of a symmetrical position of the corresponding observation position are both normal (are not abnormality candidates), the dissimilarity determination unit 114a is configured to determine whether or not the biological sound signal of the corresponding observation position and the biological sound signal of the corresponding symmetrical position are not similar. Further, in a case where the dissimilarity determination unit 114a determines that the biological sound signal of the corresponding observation position and the biological sound signal of the corresponding symmetrical position are not similar, the display control unit 113a performs indication so that re-observation is performed once (observation is performed a total of two times) for each of the corresponding observation position and the corresponding symmetrical position.

That is, if biological sound information that is obtained in the first observation of an observation position of an odd-numbered observation number (assuming a case of an observation number g) is determined to be normal by the abnormality determination unit 111a, biological sound information that is obtained in the first observation of an observation position of an even-numbered observation number (assuming a case of an observation number h) that is one plus the observation number g is also determined to be normal by the abnormality determination unit 111a, and the biological sound information of the observation number g and the biological sound information of the observation number h are determined to be dissimilar by the dissimilarity determination unit 114a, the display control unit 113a makes the operator U perform re-observation of the observation position of the observation number g by indicating an observation sequence with the observation position of the observation number g as the designated observation position. That is, by displaying the observation position of the observation number g in the observation position box 400 of the indication screen 150a, and displaying an indication that designates the operator U to press the record button 500a in the indication box 600a, the display control unit 113a makes the operator U perform re-observation of the observation position of the observation number g. Subsequently, regardless of whether or not it is determined that the biological sound information that is obtained in performing re-observation of the observation position of the observation number g is an abnormality candidate, by indicating an observation sequence with an observation position of the observation number h as the designated observation position, the display control unit 113a makes the operator U perform re-observation of the observation position of the observation number h. That is, by displaying the observation position of the observation number h in the observation position box 400a of the indication screen 150a, and displaying an indication that designates the operator U to press the record button 500a in the indication box 600a, the display control unit 113a makes the operator U perform re-observation of the observation position of the observation number h.

As a result of this, re-observation is performed for each of the observation position of the odd-numbered observation number g and the observation position (the symmetrical position of the observation number g) of the odd-numbered observation number h that is one plus the observation number g (observation is carried out a total of two times).

Subsequently, by indicating an observation sequence with an observation position of an observation number that is 1 plus the observation number h (assuming a case of an observation number i) as the designated observation position, the display control unit 113a makes the operator U perform a first observation for the observation position of the observation number i.

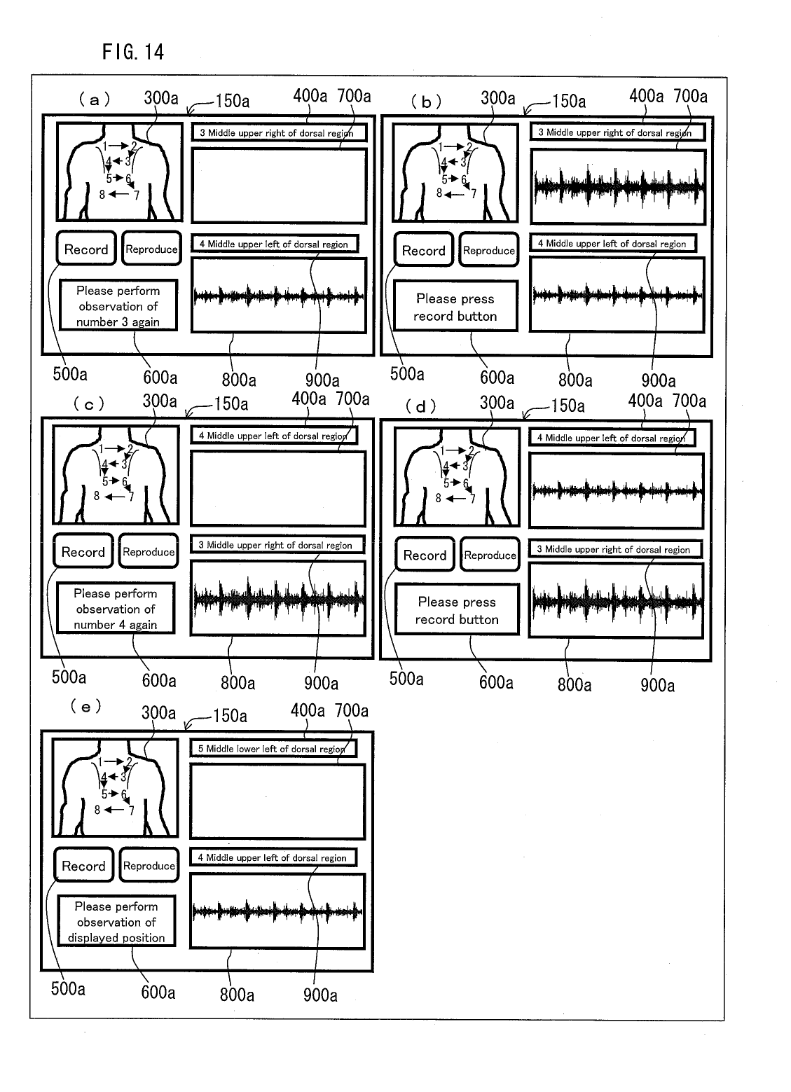

Next, an example of an indication screen that is displayed on the display unit 104a in a case where it is assumed that biological sound information of the observation number 3 and biological sound information of the observation number 4 are determined to be normal by the abnormality determination unit 111a, and the biological sound information of the observation number 3 and the biological sound information of the observation number 4 are determined to be dissimilar by the dissimilarity determination unit 114a will be described on the basis of FIGS. 13 and 14.

As has already been described using FIG. 12 and FIGS. 13(a) to 13(c), after observation of the observation numbers 1 and 2 has been finished, as shown in FIG. 13(c), by displaying the observation position of the observation number 3 in the observation position box 400a, the display control unit 113a designates (indicates) the operator U to perform a first observation of the observation position of the observation number 3. When the operator U initiates auscultation of the observation position of the observation number 3, as shown in FIG. 13(d), the display control unit 113a displays the waveform of the biological sound signal that is obtained from the observation position of the observation number 3 in the received waveform box 700a, and displays a message that designates pressing of the record button 500a in the indication box 600a. Additionally, in this instance, the biological sound information of the observation position of the observation number 3 is biological sound information that has been determined to be normal (not an abnormality candidate) by the abnormality determination unit 111a.

When the operator U presses the record button 500a in the state of FIG. 13(d), the recording process unit 112a performs recording of the biological sound information that is being received (performs recording of the biological sound information of the observation number 3). When recording is performed, as shown in FIG. 13(e), the display control unit 113a ends the waveform display of the received waveform box 700a, displays the waveform of the biological sound information of the observation number 3 that was recorded most recently in the recording waveform box 800a, and displays an observation position that corresponds to the biological sound information that was recorded in the recorded observation position box 900a.

In addition, as shown in FIG. 13(e), by displaying the observation position of the observation number 4, which is the symmetrical position of the observation position of the observation number 3, in the observation position box 400a, and displaying a message of an observation instruction for the observation number 4 in the indication box 600a, the display control unit 113a designates (indicates) the operator U to perform observation of the observation position of the observation number 4. Further, when the operator U initiates auscultation of the observation position of the observation number 4 in the same manner as that of the observation number 3, as shown in FIG. 13(f), the display control unit 113a displays a waveform of a biological sound signal that is obtained from the observation position of the observation number 4 in the received waveform box 700a, and displays a message that designates pressing of the record button 500a in the indication box 600a. Additionally, the biological sound information of the observation position of the observation number 4 is biological sound information that has been determined to be normal (not an abnormality candidate) by the abnormality determination unit 111a.

When the operator U presses the record button 500a in the state of FIG. 13(f), the recording process unit 112a performs recording of the biological sound information that is being received (the biological sound information of the observation position of the observation number 4).

In this instance, in the present embodiment, in a case where biological sound information of an odd-numbered observation number (assuming a case of an observation number j) and biological sound information of an even-numbered observation number (assuming a case of an observation number k) that is one plus the corresponding odd number are both determined to be normal, if the dissimilarity determination unit 114a determines whether or not the biological sound information of the observation number j and the biological sound information of the observation number k are not similar and the two sets of biological sound information are not similar, the display control unit 113a makes the operator U perform re-observation of the observation number j and the observation number k.

In other words, when the operator U presses the record button 500a in the state of FIG. 13(f), recording of the biological sound information of the observation position of the observation number 4 is performed, and furthermore, the dissimilarity determination unit 114a reads a biological sound signal that is recorded through acquisition thereof from the observation position of the observation number 3, and biological sound information that is recorded through acquisition thereof from the observation position of the observation number 4 from the storage unit 103a, and determines whether or not the two sets of read biological sound information are not similar.

In this instance, in a case where the dissimilarity determination unit 114a determines that the biological sound signal of the observation number 3 and the biological sound signal of the observation number 4 are not dissimilar (a case in which similarity is determined), the display control unit 113a performs indication for a first observation of the observation number 5 that is 1 plus the observation number 4 without performing indication for re-observation of the observation number 3 and the observation number 4.

In contrast to this, in a case where the dissimilarity determination unit 114a determines that the biological sound signal of the observation number 3 and the biological sound signal of the observation number 4 are not similar, the display control unit 113a is configured to indicate re-observation of the observation number 3 and the observation number 4 in order.

Therefore, in a case where the operator U presses the record button 500a and recording is performed in the state of FIG. 13(f), and furthermore, the biological sound signal of the observation number 3 and the biological sound signal of the observation number 4 are determined to be dissimilar, as shown in FIG. 14(a), by displaying the observation position of the observation number 3 in the observation position box 400a, and displaying a message of a re-observation instruction for the observation number 3 in the indication box 600a, the display control unit 113a designates (indicates) the operator U to perform re-observation of the observation position of the observation number 3.

Further, the operator U initiates re-auscultation of the observation position of the observation number 3 according to the indication screen 150a of FIG. 14(a), the communication unit 101a receives the biological sound information of the observation position of the observation number 3, and the abnormality determination unit 111a performs determination of whether or not the corresponding biological sound information is an abnormality candidate. As shown in FIG. 14(b), the display control unit 113a displays the waveform of the biological sound information of the observation number 4 that was recorded most recently in the recording waveform box 800a, and displays a message that designates pressing of the record button 500a in the indication box 600a while displaying the waveform of the observation number 3 that is being received in the received waveform box 700a.

When the operator U presses the record button 500a of the indication screen 150a that is shown in FIG. 14(b), the recording process unit 112a performs recording of the biological sound information that is being received (the biological sound information of the observation position of the observation number 3). When recording is performed, as shown in FIG. 14(c), the display control unit 113a ends the waveform display of the received waveform box 700a, displays the waveform of the biological sound information of the observation number 3 that was recorded most recently in the recording waveform box 800a, and displays an observation position that corresponds to the biological sound information that was recorded in the recorded observation position box 900a.

In addition, regardless of the determination result of the abnormality determination unit 111a with respect to the biological sound information that is obtained in the re-observation of the observation number 3, as shown in FIG. 14(c), by displaying the observation position of the observation number 4 in the observation position box 400a, and displaying a message of a re-observation instruction for the observation number 4 in the indication box 600a, the display control unit 113a designates (indicates) the operator U to perform re-observation of the observation position of the observation number 4. Further, as shown in FIGS. 14(d) and 14(e), recording is also performed in the same manner as that of the observation number 3 for the observation number 4 as a result of the record button 500a being pressed, and re-observation is completed.

In this manner, in a case where it is determined that a given biological sound signal and a biological sound signal of a symmetrical position of the corresponding observation position are normal, but are not similar to one another, re-observation is performed for each of the corresponding observation position and the corresponding symmetrical position, and as a result of this, observation is performed twice for each of the corresponding observation position and the corresponding symmetrical position.

(Process Flow)

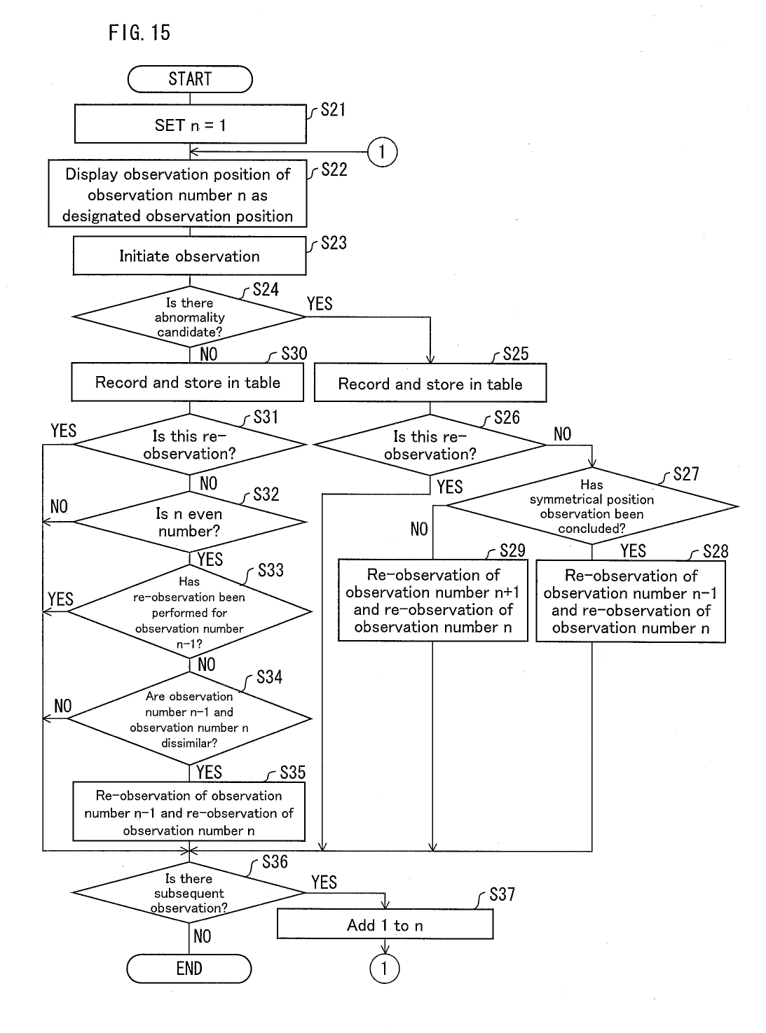

Next, an example of a process flow of Embodiment 2 will be described with reference to FIG. 15. FIG. 15 is a flowchart that shows an example of a process flow of Embodiment 2.

When the operator U inputs an execution instruction for the dorsal region mode into the terminal device 100a, the control unit 102a is configured to set n=1 (S21), set the observation position of an observation number n as the designated observation position (S22), and display the designated observation position in the observation position box 400a of the indication screen 150a.

Further, the operator U initiates auscultation (observation) of the observation position of the observation number n according to the indication of the indication box 600a of the indication screen 150a (S23). When auscultation is initiated, the control unit 102a determines whether or not the biological sound information that is obtained by the auscultation is an abnormality candidate (S24).

Processes after the control unit 102a has determined that the biological sound information is an abnormality candidate in S24 will be described.

In a case where it is determined that the biological sound information is an abnormality candidate (YES in S24), the control unit 102a records the biological sound information when the operator U presses the record button 500a of the indication screen 150a, and stores a sound data identifier, the observation number, and the abnormality sound information of the biological sound information in an observation table (S25). Additionally, in S25, since the biological sound information is an abnormality candidate, abnormality sound information of symbol O (abnormal) is configured to be stored in the observation table in association with the observation number (observation position information), the audio data identifier, and the like (refer to FIG. 11).

Next, the control unit 102a determines whether or not the processes that were performed in S23, S24 and S25 correspond to re-observation (S26). In a case where the processes correspond to re-observation (YES in S26), the control unit 102a moves the process to S36.

In contrast to this, in a case where the processes that were performed in S23, S24 and S25 do not correspond to re-observation (NO in S26), the control unit 102a determines whether or not observation of a symmetrical position with respect to the observation position of the observation number n has been concluded (S27). That is, observation of the symmetrical position has been concluded if n is an even number, and observation of the symmetrical position has not been concluded if n is an odd number.

In a case where observation of the symmetrical position with respect to the observation position of the observation number n has been concluded (YES in S27), re-observation of the observation position of an observation number n−1 and re-observation of the observation position of the observation number n are performed (S28). In other words, in S28, the same processes as those of S23, S24 and S25 (or S30) are performed with the observation position of the observation number n−1 (1 minus n) as the designated observation position, and thereafter, the same processes as those of S23, S24 and S25 (or S30) are performed with the observation position of the observation number n as the designated observation position.

In addition, in a case where it is determined, in S27, that observation of the symmetrical position with respect to the observation position of the observation number n has not been concluded (NO in S27), observation of an observation position of the observation number n+1 (a first observation), and re-observation of the observation position of the observation number n are performed (S29). In other words, in S29, the same processes as those of S23, S24 and S25 (or S30) are performed with the observation position of the observation number n+1 as the designated observation position, and thereafter, the same processes as those of S23, S24 and S25 (or S30) are performed with the observation position of the observation number n as the designated observation position.

After S28 or S29 are finished, the process moves to S36, and the control unit 102a determines whether or not there is still an observation position that should be observed next. More specifically, since there are eight observation positions in the manner that is shown in FIG. 3(a), there is not an observation position that should be observed next if n is 8 and there is an observation position that should be observed next if n is less than or equal to 7.

Therefore, in a case where n=1, the control unit 102a is configured to determine that there is still an observation position that should be observed next (YES in S36), add 1 to n (S37), and repeat the processes from S22 onwards. Further, in a case where the processes from S22 onwards are repeated and the process moves to the S36 after n has become 8, the control unit 102a is configured to determine that there is not still an observation position that should be observed next (NO in S36), and complete the process.

Next, processes after the control unit 102a has determined that the biological sound information is not an abnormality candidate in S24 will be described.

In a case where it is determined that the biological sound information is not an abnormality candidate (NO in S24), or in other words, in a case where it is determined that the biological sound information is normal, in the same manner as that of S25, the control unit 102a records the biological sound information when the operator U presses the record button 500a of the indication screen 150a, and stores a sound data identifier, the observation number, and the abnormality sound information of the biological sound information in an observation table (S30). Additionally, in S30, since the biological sound information is normal, abnormality sound information of symbol X (normal) is stored in the observation table in association with the observation number (positional information), the audio data identifier, and the like (refer to FIG. 11).

After S30, in the same manner as that of S26, the control unit 102a determines whether or not the processes that were performed in S23, S24 and S30 correspond to re-observation (S31). In a case where the processes correspond to re-observation (YES in S31), the control unit 102a moves the process to S36.

In contrast to this, in a case where the processes that were performed in S23, S24 and S30 do not correspond to re-observation (NO in S31), the control unit 102a determines whether or not n is an even number (S32). That is, the control unit 102a determines whether or not the observation number n of the observation that was performed in S23, S24 and S30 is an even number.

In a case where it is determined in S32 that n is not an even number (NO in S32), the control unit 102a moves the process to S36.

In contrast to this, in a case where it is determined in S32 that n is an even number (YES in S32), the control unit 102a determines whether or not re-observation has been performed for the observation position of the observation number n−1 (S33). Additionally, since “-(2)” is attached to observation numbers for which re-observation was performed in the table of FIG. 11, the control unit 102a is configured to be able to determine whether or not re-observation was performed by referring to the table of FIG. 11. This feature also applies to S26 and S31.

In a case where it is determined in S33 that re-observation has been performed for the observation position of the observation number n−1 (YES in S33), the control unit 102a moves the process to S36.

In contrast to this, in a case where it is determined in S33 that re-observation has not been performed for the observation position of the observation number n−1 (NO in S33), the control unit 102a determines whether or not the biological sound information that was obtained in the observation of the observation number n−1 and the biological sound information that was obtained in the observation of the observation number n are not similar (S34).

In a case where it is determined in S34 that the biological sound information that was obtained in the observation of the observation number n−1 and the biological sound information that was obtained in the observation of the observation number n are not dissimilar (NO in S34), the control unit 102a moves the process to S36.

In contrast to this, in a case where it is determined in S34 that the biological sound information that was obtained in the observation of the observation number n−1 and the biological sound information that was obtained in the observation of the observation number n are not similar (YES in S34), re-observation of the observation position of the observation number n−1 and re-observation of the observation position of the observation number n are performed (S35). In other words, in S35, the same processes as those of S23, S24 and S30 (or S25) are performed with the observation position of the observation number n−1 as the designated observation position, and thereafter, the same processes as those of S23, S24 and S30 (or S25) are performed with the observation position of the observation number n as the designated observation position.

After S35 is finished, the process moves to S36, the control unit 102a determines whether or not there is still an observation position that should be observed next, and completes the flow of FIG. 15 if there is not.

In the above-described flowchart, in a case where for example, n=2 (an even number), and it is determined that the biological sound information is an abnormality candidate in the first observation of the observation position of the observation number 2 (YES in S24), the first observation of the observation position of the observation number 2 is finished by recording the biological sound information of the observation position of the observation number 2 in S25. Thereafter, NO is selected in S26, YES is selected in S27, and in S28, re-observation of the observation position of the observation number 1 and re-observation of the observation position of the observation number 2 are performed. Subsequently, the process passes S36, n becomes 3 in S37, and the process moves to the processes of the observation number 3. In this manner, in a case where it is determined that the biological sound information of the observation position of the observation number 2 (an even number) is an abnormality candidate in the first observation, observation is performed twice for each of the observation position of the observation number 2 and the observation number 1, which is the symmetrical position of the observation number 2.

In addition, in the above-described flowchart, in a case where n=1 (an odd number), and it is determined that the biological sound information is an abnormality candidate in the first observation of the observation position of the observation number 1 (YES in S24), the first observation of the observation position of the observation number 1 is finished by recording the biological sound information of the observation position of the observation number 1 in S25. Thereafter, NO is selected in S26, NO is selected in S27, and in S29, a first observation of the observation position of the observation number 2 and re-observation of the observation position of the observation number 1 are performed. Subsequently, the process passes S36, and n becomes 2 in S37. Furthermore, the re-observation of the observation position of the observation number 2 is subsequently performed by the process passing S22, S23, S24 and S25 (or S30). Further, the process moves to S26 (S31) after S25 (or S30), and the process moves to S36 after YES has been selected in S26 (S31). After the process has moved to S36, n becomes 3 in S37, and the process moves to the processes of the observation number 3. In this manner, in a case where it is determined that the biological sound information of the observation position of the observation number 1 (an odd number) is an abnormality candidate in the first observation, observation is performed twice for each of the observation position of the observation number 1 and the observation number 2, which is the symmetrical position of the observation number 1.

In addition, in the above-described flowchart, processes after it has been determined in the first observation of the observation number 1 (n=1) in S24 that the biological sound information is not an abnormality candidate, and it has been determined in the first observation of the observation number 2 (n=2) in S24 that the biological sound information is not an abnormality candidate will be described. When it is determined for observation number 2 that the biological sound information is not an abnormality candidate in S24, the biological sound information of the observation number 2 is recorded in S30. Further, since it is the first observation of n=2, NO is selected in S31, and YES is selected in S32. Thereafter, NO is selected in S33. This is because re-observation of the observation number 1 has not been performed since it was determined in S24 in the first observation of observation number 1 that the biological sound information is not an abnormality candidate. After, NO is selected in S33, in S34, it is determined whether or not the biological sound information of observation number 1 and the biological sound information of observation number 2 (n=2) are not similar.

In S34, if it is determined that the biological sound information of observation number 1 and the biological sound information of observation number 2 (n=2) are not dissimilar (are similar) (NO in S34), the process moves to S36, n subsequently becomes 3 in S37, and the process moves to the processes of the observation number 3.

In contrast to this, in S34, if it is determined that the biological sound information of observation number 1 and the biological sound information of observation number 2 (n=2) are not similar (YES in S34), in S35, re-observation of the observation position of the observation number 1 and re-observation of the observation position of the observation number 2 are performed. Thereafter, the process passes S36, n becomes 3 in S37, and the process moves to the processes of the observation number 3. In this manner, in a case where it is determined that the biological sound information of the observation number 1 and the biological sound information of the observation number 2 that has a paired relationship with the observation number 1 are not similar, re-observation is performed for each of the observation number 1 and the observation number 2, and observation is performed a total of two times as a result.

In the manner described above, in the present embodiment the display control unit (the indication unit) 113a executes an indication process that indicates observation positions (designated observation position) on which the stethoscope 30 is to be placed at the current point in time to a user of the stethoscope 30, and the recording process unit 112a is configured to perform a biological sound observation process, which is a process for recording on the storage unit 103a by associating an observation number (positional information) that shows an observation position that is indicated by the display control unit 113a with biological sound information that is being input from the stethoscope 30. Further, in a case where the waveform of biological sound information (referred to as first biological sound information) that is recorded in the storage unit 103a in association with positional information of a given observation position (referred to as a first observation position) and the waveform of biological sound information (referred to as second biological sound information) that is recorded in the storage unit 103a in association with positional information of an observation position (referred to as a second observation position) that has a bilaterally symmetrical relationship with the first observation position are both determined to be normal by the abnormality determination unit 111a, the dissimilarity determination unit 114a is configured to determine whether or not the first biological sound information and the second biological sound information are not similar. Further, in a case where dissimilar is determined in the above-described dissimilarity determination, the display control unit 113a has a configuration that performs an indication process so that the biological sound observation process is performed again (so that re-observation is performed) for each of the first observation position and the second observation position.

According to this configuration, in a case where it is determined that the biological sound information of the first observation position and the biological sound information of the second observation position (a position that is bilaterally symmetrical to the first observation position), which has a paired relationship with the first observation position, are not similar, it becomes possible to reproduce the biological sound information of at least two auscultations for each of the first observation position and the second observation position, and therefore, there is an effect of being capable of improving the precision of the diagnosis of a doctor or the like.

In addition, in the present embodiment, the display control unit is configured to perform the indication process so that the biological sound observation process is performed at least twice for each of two observation positions that have a bilaterally symmetrical relationship in a case where biological sound information that corresponds to at least one of such observation positions is determined to be an abnormality candidate by the abnormality determination unit 111a. As a result of this, a doctor, who performs diagnosis by reproducing the biological sound information, has the opportunity to listen to the biological sound information of at least two auscultations for each of the observation position that is an abnormality candidate and an observation position that is bilaterally symmetrical with respect to the observation position, and therefore, it is possible to improve the precision of the diagnosis.

Furthermore, according to the present embodiment, in a case where, among two sets of biological sound information that are obtained from two observation positions that have a bilaterally symmetrical relationship, at least one of the sets of biological sound information is essentially abnormal, but both sets of biological sound information are mistakenly determined to be normal by the abnormality determination unit 111a, by determining that the two sets of biological sound information are not similar using the dissimilarity determination unit 114a, it is possible to perform the biological sound observation process again for the two observation positions (it is possible to implement re-observation), and therefore, it is possible to prevent a circumstance in which the biological sound observation process is concluded with a single observation of the two observation positions.

In addition, in the present embodiment, rather than merely recording the biological sound information in the storage unit 103a, the recording process unit 112a records by linking an observation number (positional information) that shows an observation position, abnormality sound information (abnormality determination information), which is a determination result of the abnormality determination unit 111a, the biological sound information and dissimilarity determination information, which is a determination result of the dissimilarity determination unit 114a. For this reason, since it is possible to recognize observation positions that require special attention by showing a correspondence relationship of the observation number, the dissimilarity determination information, and the abnormality sound information to a user or a doctor that performs diagnosis using information that is stored in the storage unit 103a, there is an effect of being capable of improving the precision of the diagnosis.

In addition, in the indication screens 150a, the display control unit 113a displays all of the observation positions (an observation position that is being designated, observation positions that have not been designated yet and observation positions that have been designated) as observation numbers on the pseudo human body image 300a. In this instance, the display control unit 113a may make the display form of observation numbers of observation positions that correspond to biological sound information that is determined to be an abnormality candidate, the display form of observation numbers of observation positions that correspond to biological sound information that is determined to be dissimilar, and the display form of observation numbers of other observation positions differ. In addition, in this format, the display form of observation numbers of symmetrical positions of observation positions that are determined to be abnormality candidates (referred to as abnormality observation positions) may be made to differ from the display form of the observation numbers of other observation positions, or may be made to be the same as the display form of the observation numbers of the abnormality observation positions. For example, the display forms may be made different by making the colors thereof different, or the display forms may be made different by the presence or absence of a flashing light. As a result of this, from the point of view of the operator U, an effect of being able to know, among all of the observation positions for which auscultation was performed, observation positions for which there is a possibility of an abnormality, at a glance, is exhibited.

In addition, the display control unit 113a may also be configured to display the table that is shown in FIG. 11 on the display unit 104a in response to an instruction from the operator U. Furthermore, in this case, in the table that is shown in FIG. 11, the display color of fields in which abnormality sound information is symbol O (an abnormality candidate), the display color of fields in which the dissimilarity information is symbol O (dissimilar), and a display color of other fields may be made to differ. In this case, from the point of view of the operator U, an effect of being able to know observation positions for which there is a possibility of an abnormality, at a glance, is exhibited.

### Modification Example 1

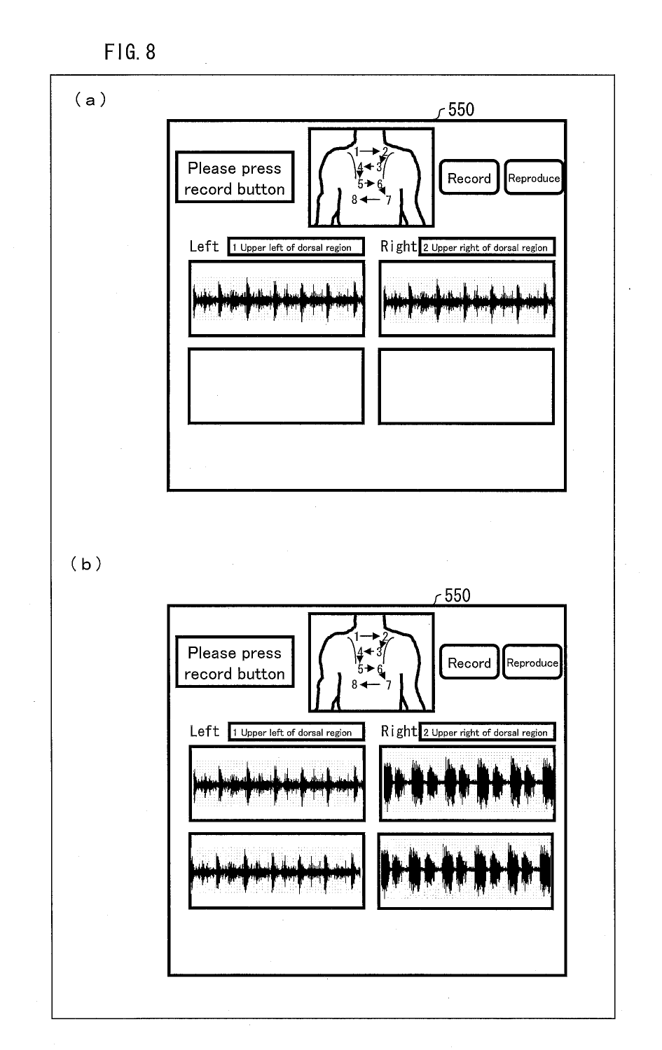

In addition, in Embodiments 1 and 2 that are described above, the display control units 113 and 113a may be configured to display indication screens 550 such as those shown in FIG. 8 on the display units 104 and 104a. In the indication screens 550 of FIG. 8, it is possible to display a waveform of the biological sound information that has been recorded during the first observation and a waveform of the biological sound information that has been recorded during the re-observation for each of a given observation position and a symmetrical position that have a paired relationship. In other words, it is possible to display four waveforms simultaneously.

More specifically, a unique indication screen 550 is displayed for each group of a given observation position and a symmetrical position that have a paired relationship. Further, the indication screen 550 is configured so that each time a recording operation is carried out, the waveform of the biological sound information that was recorded is sequentially displayed. That is, FIG. 8 illustrates unique indication screens 550 of a group of the observation number 1 and the observation number 2. In particular, FIG. 8(a) is an indication screen 550 after the first observation of the observation position of the observation number 1 and the first observation of the observation position of the observation number 2 have been completed, and displays a waveform of the biological sound information during the first observation of the observation position of the observation number 1 and a waveform of the biological sound information during the first observation of the observation position of the observation number 2. In contrast to this, and FIG. 8(b) is an indication screen 550 after the re-observation of the observation position of the observation number 1 and the re-observation of the observation position of the observation number 2 have been completed, displays the waveform of the biological sound information during the first observation and the waveform of the biological sound information during the re-observation for the observation position of the observation number 1 and a waveform of the biological sound information during the first observation and the waveform of the biological sound information during the re-observation for the observation position of the observation number 2.

As described above, in indication screens 550 such as those shown in FIG. 8, the waveform of the biological sound information that corresponds to a given observation position (an observation position α) and the waveform of the biological sound information that corresponds to a given observation position (an observation position β) are shown simultaneously. For this reason, there is an advantage in that comparison work of the waveform of the biological sound information of the given observation position and the waveform of the biological sound information of the symmetrical position is facilitated.

In addition, in an indication screen 550 such as that of FIG. 8 in a case where observation of the given observation position and the symmetrical position that have a paired relationship is performed twice, it is possible to simultaneously display the waveforms of four sets of biological sound information that are recorded in these observations, and therefore, an operator U can also perform the work of comparing the four waveforms easily.

In addition, according to the indication screens 550 that are shown in FIG. 8, among observation positions that have a bilaterally symmetrical relationship, the waveform of the biological sound information of the observation position on the left side is displayed on the left side, and the waveform of the biological sound information of the observation position on the right side is displayed on the right side, and therefore, there is an advantage in that an operator U can know whether or not a waveform is a waveform of the observation position of the left side or the right side, at a glance.

### Modification Example 2

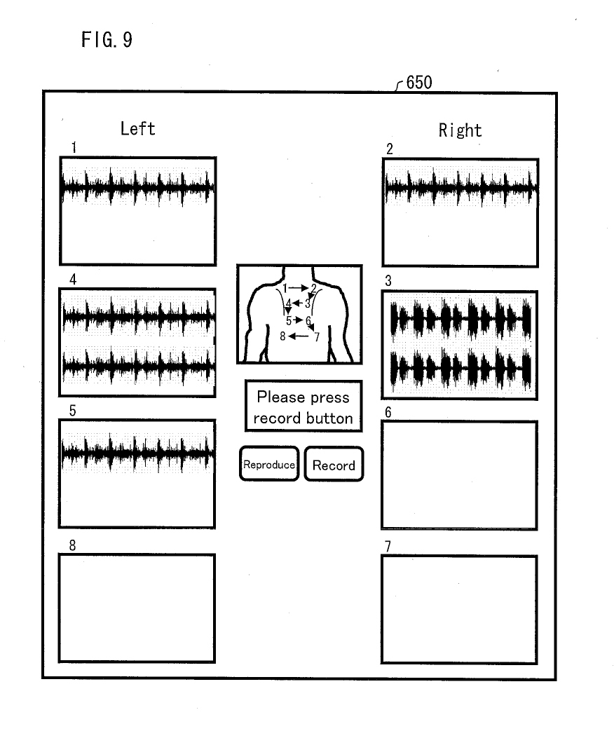

In addition, in Embodiments 1 and 2 that are described above, the display control units 113 and 113a may be configured to display an indication screen 650 such as that shown in FIG. 9 on the display units 104 and 104a. In the indication screen 650 of FIG. 9, a box that displays the waveform of the biological sound information of each observation position for which recording has been completed is shown for each observation position, and each box shows a waveform of the biological sound information that has been recorded during the first observation and a waveform of the biological sound information that has been recorded during the re-observation.

That is, in the indication screen 650 that is shown in FIG. 9, it is possible to collectively confirm waveforms of the biological sound information for all of the observation positions. Furthermore, in the indication screen 550 that is shown in FIG. 9, among observation positions that have a bilaterally symmetrical relationship, the waveform of the biological sound information of the observation position on the left side is displayed on the left side, and the waveform of the biological sound information of the observation position on the right side is displayed on the right side, and therefore, there is an advantage in that an operator U can know whether or not a waveform is a waveform of the observation position of the left side or the right side, at a glance.

[Implementation Example Using Software]

Finally, each block of the control units 102 and 102a may be realized in hardware using a logic circuit that is formed on an integrated circuit (IC chip), or may be realized as software using a Central Processing Unit (CPU).

In a case of the latter, the control units 102 and 102a are provided with a CPU that executes orders of programs that realize each function, Read Only Memory (ROM) that stores the above-described programs, Random Access Memory (RAM) that deploys the above-described programs, and a memory device (recording medium) such as memory or the like that stores the above-described programs and various kinds of data. Further, the object of the present invention can also be achieved by supplying a recording medium in which program code (an executable program, an intermediate code program, of a source program) of the control programs of the control units 102 and 102a, which is are pieces of software that realize the above-described functions, is stored so as to be readable by a computer, to the control units 102 and 102a, and the computer (alternatively, a CPU or an MPU) reading and executing the program code that is stored in the recording medium.

It is possible to use a non-transitory tangible medium, for example, a tape such as a magnetic tape or a cassette tape, a disk that includes magnetic disks such as a floppy (registered trademark) disk/ or a hard disk, or an optical disk such as a CD-ROM/MO/MD/DVD/ or a CD-R, a card such as an IC card (including a memory card)/ or an optical card, semiconductor memory such as a mask ROM/EPROM/EEPROM (registered trademark)/ or flash ROM, or a logic circuit such as a Programmable Logic Device (PLD) or a Field Programmable Gate Array (FPGA) as the above-described recording medium.

In addition, the control units 102 and 102a may be configured to be capable of connecting to a communications network, and supplying the above-described program code via the communications network. The communications network is not particularly limited as long as it is capable of transmitting the program code. For example, the Internet, an intranet, an extranet, a LAN, an ISDN, a VAN, a CATV communication network, a virtual private network, a telephone line network, a mobile communication network, a satellite communication network, or the like can be used. In addition, as long as the transmission medium that configures the communication network, and the program code are media that are capable of being transmitted, the above-described components are not limited to a particular configuration or kind. For example, use in wired communication such as IEEE1394, USB, a power-line carrier, a cable TV line, a telephone line, an Asymmetric Digital Subscriber Line (ADSL) or the like, and wired communication such as infrared radiation using IrDA of a remote control, Bluetooth (registered trademark), IEEE802.11 wireless, High Data Rate (HDR), Near Field Communication (NFC), Digital Living Network Alliance (DLNA), a mobile phone network, a satellite connection, a terrestrial digital network or the like, is possible.

The present invention is not limited to the embodiments described above, various modifications are possible within the scope of the claims, and embodiments that are obtained by combining the technical means that are respectively disclosed in different embodiments are also included in the technical scope of the present invention.

### Summary of Embodiment 1

As described in Embodiment 1, the information processing device according to the first embodiment of the present invention (the terminal device), is provided with an indication unit that indicates observation positions on which a stethoscope is to be placed to a user of the stethoscope, a recording process unit that performs a biological sound observation process, which is a process for recording on a storage device by associating positional information that shows observation positions which are indicated by the indication unit with biological sound information that is received by the stethoscope, and an abnormality determination unit that determines whether or not the biological sound information is an abnormality candidate. When the indication unit sets an observation position that is shown by positional information which is associated with biological sound information that is determined to be an abnormality candidate as an abnormality observation position, and sets an observation position, which has a paired relationship with the abnormality observation position according to a predetermined rule, as a predetermined observation position in a case where it is determined in the abnormality determination unit that the biological sound information is an abnormality candidate, the indication unit performs the indication so that the biological sound observation process is executed at least twice for each of the abnormality observation position and the predetermined observation position.

According to this configuration, in a case where biological sound information for which there is a suspicion of an abnormality, is acquired (a case in which the biological sound information is determined to be an abnormality candidate), the biological sound observation process is performed at least twice for each of the abnormality observation position, which is an acquisition source of the corresponding biological sound information, and the predetermined observation position (a symmetrical position), which has a paired relationship with the abnormality observation position according to a predetermined rule. Therefore, since a doctor, who subsequently performs diagnosis by reproducing the biological sound information, has the opportunity to listen to at least two items of biological sound information for each of the abnormality observation position for which there is a suspicion of an abnormality and the predetermined observation position, there is an effect of being capable of improving the precision of the diagnosis.

In addition, as shown in Embodiment 1, it is preferable that the abnormality determination unit be configured so as to output abnormality sound information that shows whether or not the biological sound information is an abnormality candidate, and the recording process unit record on the memory device by associating the positional information with the biological sound information and the abnormality sound information.

According to this configuration, it is possible to read the positional information of an observation position which is an acquisition source of the biological sound information, and the abnormality sound information that shows whether or not the biological sound information is an abnormality candidate and perform display thereof or the like to a user or a doctor. For this reason, since it is possible to alert a user or a doctor who attempts to reproduce the biological sound information of an abnormality candidate that there is a possibility of an abnormality in advance, the user or the doctor listens to the biological sound information carefully, and therefore, there is an effect of being capable of improving the precision of the diagnosis.

In addition, in a case where diagnosis of a given observation position is performed, it is important to compare the biological sound information of the corresponding observation position and the biological sound information of a symmetrical position of the corresponding observation position rather than just evaluating the biological sound information of the corresponding observation position only. In such an instance, it is preferable that the information processing apparatus be provided with a display control unit that displays a waveform of biological sound information that corresponds to one observation position among two observation positions that has a paired relationship according to a predetermined rule, and a waveform of biological sound information that corresponds to the other observation position among the two observation positions simultaneously. In addition, it is preferable that the information processing apparatus be provided with a display control unit that displays a waveform of biological sound information that is associated with the positional information of a first observation position, and a waveform of biological sound information that is associated with the positional information of a second observation position that has a paired relationship with the first observation position according to a predetermined rule simultaneously. For this reason, there is an advantage in that comparison work of the waveform of the biological sound information of the first observation position and the waveform of the biological sound information of the second observation position (the symmetrical position) is facilitated.

In addition, the indication unit may display an image in which observation positions for which indication has been completed and observation positions for which indication has not been completed are shown on a pseudo human body image, and may make the display form of the abnormality observation positions and the display form of observation positions other than the abnormality observation positions differ in the image.

According to this configuration, an effect of being able to know observation positions for which there is a possibility of an abnormality, at a glance, is exhibited.

In addition, the indication unit may display an image in which observation positions for which indication has been completed and observation positions for which indication has not been completed are shown on a pseudo human body image, and may make the display form of the abnormality observation positions and the predetermined observation positions, and the display form of observation positions other than the abnormality observation positions and the predetermined observation positions differ in the image.

According to this configuration, from the point of view of a user, an effect of being able to know, among all of the observation positions for which auscultation was performed, the abnormality observation positions and the predetermined observation positions for which the biological sound observation process was performed at least twice at a glance, is exhibited.

In addition, the first embodiment of the present invention may be an information processing method. The information processing method includes an indication step in which the information processing device indicates observation positions on which a stethoscope is to be placed to a user of the stethoscope, a recording step in which the information processing device performs a biological sound observation process, which is a process for recording on a storage device by associating positional information that shows observation positions which are indicated in the indication step by the indication unit with biological sound information that is received by the stethoscope, and a determination step in which the information processing device determines whether or not the biological sound information is an abnormality candidate. In the indication step, when the information processing device sets an observation position that is shown by positional information which is associated with biological sound information that is determined to be an abnormality candidate as an abnormality observation position, and sets an observation position, which has a paired relationship with the abnormality observation position according to a predetermined rule, as a predetermined observation position in a case where it is determined in the recording step that the biological sound information is an abnormality candidate, the information processing device performs the indication so that the biological sound observation process is executed at least twice for each of the abnormality observation position and the predetermined observation position.

Accordingly, since a doctor, who subsequently performs diagnosis by reproducing the biological sound information, has the opportunity to listen to at least two items of biological sound information for each of the abnormality observation position for which there is a suspicion of an abnormality and the predetermined observation position that has a paired relationship with the corresponding observation position, there is an effect of being capable of improving the precision of the diagnosis.

Furthermore, the information processing device may be realized by a computer, and in this case, programs that cause a computer function as each unit of the information processing device, and recording medium, which are readable by the computer, on which the programs are stored are also included in the category of the present invention.

### Summary of Embodiment 2

As described in Embodiment 2, the information processing device according to the second embodiment is provided with an indication unit that indicates observation positions on which a stethoscope is to be placed to a user of the stethoscope, a recording process unit that performs a biological sound observation process, which is a process for recording on a storage device by associating positional information that shows observation positions which are indicated by the indication unit with biological sound information that is received by the stethoscope, and a dissimilarity determination unit that performs dissimilarity determination of whether or not a waveform of first biological sound information that is stored in the storage device in association with positional information of a first observation position, and a waveform of second biological sound information that is stored in the storage device in association with a second observation position, which is an observation position that has a paired relationship with the first observation position according to a predetermined rule, are not similar. In a case where dissimilarity is determined in the dissimilarity determination, the indication unit is configured to perform the indication process such that the biological sound observation process is performed again for each of the first observation position and the second observation position.

According to this configuration, in a case where there is a possibility that the biological sound information of the first observation position and the biological sound information of the second observation position (a position that is bilaterally symmetrical to the first observation position), which has a paired relationship with the first observation position, are not similar, it is possible to reproduce the biological sound information of at least two auscultations for each of the first observation position and the second observation position, and therefore, there is an effect of being capable of improving the precision of the diagnosis.

Further, in addition to the above configuration, the information processing device is provided with an abnormality determination unit that performs an abnormality determination of whether or not the biological sound information that is received by the stethoscope is an abnormality candidate. In a case where it is determined that biological sound information that is stored in the memory device in association with positional information of at least one observation position of two observation positions that have a paired relationship according to a predetermined rule, the indication unit performs the indication so that the biological sound observation process is performed again for each of the two observation positions, and in a case where it is determined by the abnormality determination unit that both biological sound information that is stored in the memory device in association with positional information of an observation position of the two observation positions, and biological sound information that is stored in the memory device in association with positional information of the other observation position of the two observation positions are not abnormality candidates, the dissimilarity determination unit performs dissimilarity determination with one of the two observation positions set as the first observation position and the other of the two observation positions set as the second observation position.

According to this configuration, in a case where biological sound information for which there is a suspicion of an abnormality for at least one of two observation positions that have a paired relationship according to a predetermined rule, is acquired (a case in which the observation position is determined as an abnormality candidate), the biological sound observation process is performed at least twice for each of the two observation positions. Therefore, since a doctor, who subsequently performs diagnosis by reproducing the biological sound information, has the opportunity to listen to the biological sound information of at least two auscultations for each of the abnormality observation position which is an abnormality candidate and the observation position that has a paired relationship with the abnormality observation position according to a predetermined rule, there is an effect of being capable of improving the precision of the diagnosis.

Furthermore, according to this configuration, in a case where, among two sets of biological sound information that are obtained from two observation positions that have a paired relationship according to a predetermined rule, at least one of the sets of biological sound information is essentially abnormal, but both sets of biological sound information are mistakenly determined to be normal by the abnormality determination unit, by determining that one of the sets of biological sound information is dissimilar to the other using the dissimilarity determination unit, it is possible to implement the biological sound observation process again for the two observation positions, and therefore, it is possible to prevent a circumstance in which the biological sound observation process is concluded with a single observation of the two observation positions.

In addition, the recording process unit may record on the memory device by associating dissimilarity determination information that shows a determination result of the dissimilarity determination with each of the positional information of the first observation position and the positional information of the second observation position.

According to this configuration, since it is possible to recognize observation positions that require special attention (or in other words, sets of biological sound information that are determined to be dissimilar in the dissimilarity determination) by showing a correspondence relationship of the positional information and the dissimilarity determination information to a user or a doctor that performs diagnosis using information that is stored in the memory device, there is an effect of being capable of improving the precision of the diagnosis.

In addition, the recording process unit may record on the memory device by associating abnormality determination information that shows a determination result of the abnormality determination of the biological sound information that is associated with each item of positional information with each item of positional information that is stored in the memory device.

According to this configuration, since it is possible to recognize observation positions that require special attention (or in other words, biological sound information that is determined to be an abnormality candidate in the abnormality determination) by showing a correspondence relationship of the positional information and the abnormality determination information to a user or a doctor that performs diagnosis using information that is stored in the memory device, there is an effect of being capable of improving the precision of the diagnosis.

In addition, the second embodiment of the present invention may be an information processing method. The information processing method includes a step in which an indication unit that executes an indication process that indicates observation positions on which a stethoscope is to be placed to a user of the stethoscope, a step in which a recording process unit performs a biological sound observation process, which is a process for recording on a storage device by associating positional information that shows observation positions which are indicated by the indication unit with biological sound information that is received by the stethoscope, and a step in which a dissimilarity determination unit performs determination of whether or not a waveform of first biological sound information that is stored in the storage device in association with positional information of a first observation position, and a waveform of second biological sound information that is stored in the storage device in association with a second observation position, which is an observation position that has a paired relationship with the first observation position according to a predetermined rule, are not similar. In a case where it is determined by the dissimilarity determination that the first biological sound information and the second biological sound information are not similar, the indication unit performs the indication process such that the biological sound observation process is performed again for each of the first observation position and the second observation position.

As a result of this, in a case where there is a possibility that the biological sound information of the first observation position and the biological sound information of the second observation position (a position that is bilaterally symmetrical to the first observation position), which has a paired relationship with the first observation position, are not similar, it is possible to reproduce the biological sound information of at least two auscultations for each of the first observation position and the second observation position, and therefore, there is an effect of being capable of improving the precision of the diagnosis.

Furthermore, the information processing device may be realized by a computer, and in this case, programs that cause a computer function as each unit of the information processing device, and recording medium, which are readable by the computer, on which the programs are stored are also included in the category of the present invention.

### INDUSTRIAL APPLICABILITY

The present invention can be used in an information processing device that processes and managers biological sound information that is output from an electronic stethoscope.

### REFERENCE SIGNS LIST

30 stethoscope
100, 100a terminal device
102, 102a control unit
103, 103a storage unit
104, 104a display unit
111, 111a abnormality determination unit
112, 112a recording process unit
113, 113a display control unit
114a dissimilarity determination unit
150, 150a indication screen
200 auscultation system
300, 300a pseudo human body image

1-14. (canceled)

15. An information processing device comprising:
an indication unit that indicates observation positions on which a stethoscope is to be placed to a user of the stethoscope;
a recording process unit that performs a biological sound observation process, which is a process for recording on a storage device by associating positional information that shows observation positions which are indicated by the indication unit with biological sound information that is received by the stethoscope; and
an abnormality determination unit that determines whether or not the biological sound information is an abnormality candidate,
wherein, when the indication unit sets an observation position that is shown by positional information which is associated with biological sound information that is determined to be an abnormality candidate as an abnormality observation position, and sets an observation position, which has a paired relationship with the abnormality observation position according to a predetermined rule, as a predetermined observation position in a case where it is determined in the abnormality determination unit that the biological sound information is an abnormality candidate, the indication unit performs the indication such that the biological sound observation process is executed at least twice for each of the abnormality observation position and the predetermined observation position.

16. The information processing device according to claim 15,
wherein the abnormality determination unit outputs abnormality sound information that shows whether or not the biological sound information is an abnormality candidate, and
the recording process unit records on the storage device the positional information, the biological sound information, and the abnormality sound information that are associated with each other.

17. The information processing device according to claim 15, further comprising:
a display control unit that simultaneously displays on a display device a waveform of biological sound information that is associated with a first observation position of two observation positions and a waveform of biological sound information that is associated with a second observation position of the two observation positions, the first and the second observation positions having a paired relationship according to the predetermined rule.

18. The information processing device according to claim 15,
wherein the indication unit displays an image on a display device that indicates observation positions having been observed and observation positions to be observed, on a simulated human body image such that an abnormality observation position is shown in a manner different from that of the other observation positions.

19. The information processing device according to claim 15,
wherein the indication unit displays an image on a display device that indicates observation positions having been observed and observation positions to be observed, on a simulated human body image such that a pair of an abnormality observation position and a predetermined observation position that has a paired relationship with the abnormality observation position are shown in a manner different from that of the other observation positions.

20. The information processing device according to claim 15, further comprising:
a dissimilarity determination unit that, in a case where it is determined by the abnormality determination unit that both first biological sound information that is stored in the storage device in association with positional information of a first observation position, and second biological sound information that is stored in the storage device in association with a second observation position, which is an observation position that has a paired relationship with the first observation position according to a predetermined rule, are not abnormality candidates, performs dissimilarity determination of whether or not the first biological sound information, and the second biological sound information are not similar,
wherein, in a case where it is determined by the dissimilarity determination that the first biological sound information and the second biological sound information are not similar, the indication unit is configured to perform the indication process such that the biological sound observation process is performed again for each of the first observation position and the second observation position.

21. An information processing device comprising:
an indication unit that executes an indication process that indicates observation positions on which a stethoscope is to be placed to a user of the stethoscope;
a recording process unit that performs a biological sound observation process, which is a process for recording on a storage device by associating positional information that shows observation positions which are indicated by the indication unit with biological sound information that is received by the stethoscope; and
a dissimilarity determination unit that performs dissimilarity determination of whether or not a waveform of first biological sound information that is stored in the storage device in association with positional information of a first observation position, and a waveform of second biological sound information that is stored in the storage device in association with a second observation position, which is an observation position that has a paired relationship with the first observation position according to a predetermined rule, are not similar,
wherein, in a case where it is determined by the dissimilarity determination that the first biological sound information and the second biological sound information are not similar, the indication unit is configured to perform the indication process such that the biological sound observation process is performed again for each of the first observation position and the second observation position.

22. The information processing device according to claim 21, further comprising:
an abnormality determination unit that performs an abnormality determination of whether or not biological sound information that is received by the stethoscope is an abnormality candidate,
wherein the indication unit is a unit that performs the indication process such that the biological sound observation process is performed again for each of two observation positions in a case where it is determined by the abnormality determination unit that biological sound information, which is stored in the storage device in association with positional information of at least one observation position of the two observation positions that have a paired relationship according to a predetermined rule, is an abnormality candidate, and
wherein the dissimilarity determination unit performs the above-described dissimilarity determination with a first of the two observation positions as a first observation position and a second of the two observation positions as a second observation position in a case where it is determined by the abnormality determination unit that both of biological sound information that is stored in the storage device in association with positional information of the first observation position of the two observation positions, and biological sound information that is stored in the storage device in association with positional information of the second observation position of the two observation positions are not abnormality candidates.

23. The information processing device according to claim 21,
wherein the recording process unit records on the storage device by associating dissimilarity determination information that shows a determination result of the dissimilarity determination with each item of positional information of the first observation position and positional information of the second observation position.

24. The information processing device according to claim 22,
wherein the recording process unit records on the storage device by associating abnormality determination information, which shows a determination result of the abnormality determination of biological sound information that is associated with each item of positional information, with each item of positional information that is stored in the storage device.

25. A control method of an information processing device comprising:
an indication step in which the information processing device indicates observation positions on which a stethoscope is to be placed to a user of the stethoscope;
a recording step in which the information processing device performs a biological sound observation process, which is a process for recording on a storage device by associating positional information that shows observation positions which are indicated in the indication step with biological sound information that is received by the stethoscope; and
a determination step in which the information processing device determines whether or not the biological sound information is an abnormality candidate,
wherein, in the indication step, when the information processing device sets an observation position that is shown by positional information which is associated with biological sound information that is determined to be an abnormality candidate as an abnormality observation position, and sets an observation position, which has a paired relationship with the abnormality observation position according to a predetermined rule, as a predetermined observation position in a case where it is determined in the recording step that the biological sound information is an abnormality candidate, the information processing device performs the indication such that the biological sound observation process is executed at least twice for each of the abnormality observation position and the predetermined observation position.

26. A control method of an information processing device comprising:
a step in which an indication unit executes an indication process that indicates observation positions on which a stethoscope is to be placed to a user of the stethoscope;
a step in which a recording process unit performs a biological sound observation process, which is a process for recording on a storage device by associating positional information that shows observation positions which are indicated by the indication unit with biological sound information that is received by the stethoscope; and
a step in which a dissimilarity determination unit performs dissimilarity determination of whether or not a waveform of first biological sound information that is stored in the storage device in association with positional information of a first observation position, and a waveform of second biological sound information that is stored in the storage device in association with a second observation position, which is an observation position that has a paired relationship with the first observation position according to a predetermined rule, are not similar,
wherein, in a case where it is determined by the dissimilarity determination that the first biological sound information and the second biological sound information are not similar, the indication unit is configured to perform the indication process such that the biological sound observation process is performed again for each of the first observation position and the second observation position.

27. A non-transitory computer-readable recording medium in which a program that causes a computer to function as each unit of the information processing device according to claim 15 is stored.

28. A non-transitory computer-readable recording medium in which a program that causes a computer to function as each unit of the information processing device according to claim 21 is stored.
